SEQUENCE LISTING (3 sequences)

SEQ ID NO: 1 - DNA, 2046 bp, synthetic construct
  source 1..2046: mol_type = other DNA
atgaagaccacatgtttccttatcagtcttatcttaattcaagggacaaaaaatctccccattttagagatagctagtaataatcaaccccaaaatgtggattcggtatgctccggaactctccagaagacagaagacgtccatctgatgggattcacactgagtgggcaaaaagttgctgattcccctttggaggcatccaagcgatgggctttcaggacaggtgtacctcccaagaatgttgagtacacagagggggaggaagccaaaacatgctacaatataagtgtaacggatccctctggaaaatccttgctgttagatcctcctaccaacatccgtgactatcctaaatgcaaaactatccatcatattcaaggtcaaaaccctcatgcacaggggatcgcccttcatttatggggagcattttttctgtatgatcgcattgcctccacaacaatgtaccgaggcaaagtcttcactgaagggaacatagcagctatgattgtcaataagacagtgcacaaaatgattttctcgcggcaaggacaagggtaccgtcatatgaatctgacttctactaataaatattggacaagtagtaacggaacgcaaacgaatgacactggatgtttcggcgctcttcaagaatacaattctacaaagaaccaaacatgtgctccgtccaaaatacctccaccactgcccacagcccgtccggagatcaaactcacaagcaccccaactgatgccaccaaactcaataccacggacccaagcagtgatgatgaggacctcgcaacatccggctcagggtccggagaacgagaaccccacacaacttctgatgcggtcaccaagcaagggctttcatcaacaatgccacccactccctcaccacaaccaagcacgccacagcaaggaggaaacaacacaaaccattcccaagatgctgtgactgaactagacaaaaataacacaactgcacaaccgtccatgccccctcataacactaccacaatctctactaacaacacctccaaacacaacttcagcactctctctgcaccattacaaaacaccaccaatgacaacacacagagcacaatcactgaaaatgagcaaaccagtgccccctcgataacaaccctgcctccaacgggaaatcccaccacagcaaagagcaccagcagcaaaaaaggccccgccacaacggcaccaaacacgacaaatgagcatttcaccagtcctccccccacccccagctcgactgcacaacatcttgtatatttcagaagaaagcgaagtatcctctggagggaaggcgacatgttcccttttctggatgggttaataaatgctccaattgattttgacccagttccaaatacaaaaacaatctttgatgaatcctctagttctggtgcctcggctgaggaagatcaacatgcctcccccaatattagtttaactttatcttattttcctaatataaatgagaacactgcctactctggagaaaatgagaatgattgtgatgcagagttaagaatttggagcgttcaggaggatgacctggccgcagggctcagttggataccgttttttggccctggaattgaaggactttacactgctgttttaattaaaaatcaaaacaatttggtctgcaggttgaggcgtctagccaatcaaactgccaaatccttggaactcttattgagagtcacaactgaggaaagaacattctccttaatcaatagacatgctattgactttctactcacaagatggggaggaacatgcaaagtgcttggacctgattgttgcatcgggatagaagacttgtccaaaaatatttcagagcaaattgaccaaattaaaaaggacgaacaaaaagaggggactggttggggtctgggtggtaaatggtggacatccgactggggtgttcttactaacttgggcattttgctactattatccatagctgtcttgattgctctatcctgtatttgtcgtatctttactaaatatatcggataa

SEQ ID NO: 2 - DNA, 11658 bp, synthetic construct
  source 1..11658: mol_type = other DNA
acgaagacaaacaaaccattattatcattaaaaggctcaggagaaactttaacagtaatcaaaatgtctgttacagtcaagagaatcattgacaacacagtcgtagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgctacatatgaaaaaaactaacagatatcatggataatctcacaaaagttcgtgagtatctcaagtcctattctcgtctggatcaggcggtaggagagatagatgagatcgaagcacaacgagctgaaaagtccaattatgagttgttccaagaggatggagtggaagagcatactaagccctcttattttcaggcagcagatgattctgacacagaatctgaaccagaaattgaagacaatcaaggcttgtatgcaccagatccagaagctgagcaagttgaaggctttatacaggggcctttagatgactatgcagatgaggaagtggatgttgtatttacttcggactggaaacagcctgagcttgaatctgacgagcatggaaagaccttacggttgacatcgccagagggtttaagtggagagcagaaatcccagtggctttcgacgattaaagcagtcgtgcaaagtgccaaatactggaatctggcagagtgcacatttgaagcatcgggagaaggggtcattatgaaggagcgccagataactccggatgtatataaggtcactccagtgatgaacacacatccgtcccaatcagaagcagtatcagatgtttggtctctctcaaagacatccatgactttccaacccaagaaagcaagtcttcagcctctcaccatatccttggatgaattgttctcatctagaggagagttcatctctgtcggaggtgacggacgaatgtctcataaagaggccatcctgctcggcctgagatacaaaaagttgtacaatcaggcgagagtcaaatattctctgtagactatgaaaaaaagtaacagatatcacgatctaagtgttatcccaatccattcatcatgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacgctcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggactctatcggccacttcaaatgagctagtctaacttctagcttctgaacaatccccggtttactcagtctcccctaattccagcctctcgaacaactaatatcctgtcttttctatccctatgaaaaaaactaacagagatcgatctgtttacgcgctagtggatcctactcgagaacatgaagaccacatgtttccttatcagtcttatcttaattcaagggacaaaaaatctccccattttagagatagctagtaataatcaaccccaaaatgtggattcggtatgctccggaactctccagaagacagaagacgtccatctgatgggattcacactgagtgggcaaaaagttgctgattcccctttggaggcatccaagcgatgggctttcaggacaggtgtacctcccaagaatgttgagtacacagagggggaggaagccaaaacatgctacaatataagtgtaacggatccctctggaaaatccttgctgttagatcctcctaccaacatccgtgactatcctaaatgcaaaactatccatcatattcaaggtcaaaaccctcatgcacaggggatcgcccttcatttatggggagcattttttctgtatgatcgcattgcctccacaacaatgtaccgaggcaaagtcttcactgaagggaacatagcagctatgattgtcaataagacagtgcacaaaatgattttctcgcggcaaggacaagggtaccgtcatatgaatctgacttctactaataaatattggacaagtagtaacggaacgcaaacgaatgacactggatgtttcggcgctcttcaagaatacaattctacaaagaaccaaacatgtgctccgtccaaaatacctccaccactgcccacagcccgtccggagatcaaactcacaagcaccccaactgatgccaccaaactcaataccacggacccaagcagtgatgatgaggacctcgcaacatccggctcagggtccggagaacgagaaccccacacaacttctgatgcggtcaccaagcaagggctttcatcaacaatgccacccactccctcaccacaaccaagcacgccacagcaaggaggaaacaacacaaaccattcccaagatgctgtgactgaactagacaaaaataacacaactgcacaaccgtccatgccccctcataacactaccacaatctctactaacaacacctccaaacacaacttcagcactctctctgcaccattacaaaacaccaccaatgacaacacacagagcacaatcactgaaaatgagcaaaccagtgccccctcgataacaaccctgcctccaacgggaaatcccaccacagcaaagagcaccagcagcaaaaaaggccccgccacaacggcaccaaacacgacaaatgagcatttcaccagtcctccccccacccccagctcgactgcacaacatcttgtatatttcagaagaaagcgaagtatcctctggagggaaggcgacatgttcccttttctggatgggttaataaatgctccaattgattttgacccagttccaaatacaaaaacaatctttgatgaatcctctagttctggtgcctcggctgaggaagatcaacatgcctcccccaatattagtttaactttatcttattttcctaatataaatgagaacactgcctactctggagaaaatgagaatgattgtgatgcagagttaagaatttggagcgttcaggaggatgacctggccgcagggctcagttggataccgttttttggccctggaattgaaggactttacactgctgttttaattaaaaatcaaaacaatttggtctgcaggttgaggcgtctagccaatcaaactgccaaatccttggaactcttattgagagtcacaactgaggaaagaacattctccttaatcaatagacatgctattgactttctactcacaagatggggaggaacatgcaaagtgcttggacctgattgttgcatcgggatagaagacttgtccaaaaatatttcagagcaaattgaccaaattaaaaaggacgaacaaaaagaggggactggttggggtctgggtggtaaatggtggacatccgactggggtgttcttactaacttgggcattttgctactattatccatagctgtcttgattgctctatcctgtatttgtcgtatctttactaaatatatcggataataagctagctgtttacgcgttatccatgctcaaagaggcctcaattatatttgagtttttaatttttatgaaaaaaactaacagcaatcatggaagtccacgattttgagaccgacgagttcaatgatttcaatgaagatgactatgccacaagagaattcctgaatcccgatgagcgcatgacgtacttgaatcatgctgattacaacctgaattctcctctaattagtgatgatattgacaatttaatcaggaaattcaattctcttccaattccctcgatgtgggatagtaagaactgggatggagttcttgagatgttaacgtcatgtcaagccaatcccatcccaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggagtatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccaggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaatttcagactccttggccccaatcgggaactggatcagatctctggaattggtccgaaaccaagttcgtctaaatccattcaatgagatcttgttcaatcagctatgtcgtacagtggataatcatttgaaatggtcaaatttgcgaagaaacacaggaatgattgaatggatcaatagacgaatttcaaaagaagaccggtctatactgatgttgaagagtgacctacacgaggaaaactcttggagagattaaaaaatcatgaggagactccaaactttaagtatgaaaaaaactttgatccttaagaccctcttgtggtttttattttttatctggttttgtggtcttcgt

SEQ ID NO: 3 - DNA, 19 bp, synthetic construct
  source 1..19: mol_type = other DNA
cccataaggtcaccctctt

(19) United States
(12) Patent Application Publication
(10) Pub. No.: US 2025/0302896 A1
(43) Pub. Date: Oct. 2, 2025

(54) VESICULAR STOMATITIS VIRUS MARBURG VIRUS VACCINE

(71) Applicant: International AIDS Vaccine Initiative, Inc., New York, NY (US)

(72) Inventors: Christopher L. PARKS, New York, NY (US); Maoli YUAN, New York, NY (US); Christopher L. COOPER, New York, NY (US); Mark B. FEINBERG, New York, NY (US); John W. COLEMAN, New York, NY (US); Eddy SAYEED, New York, NY (US)

(21) Appl. No.: 19/063,559

(22) Filed: Feb. 26, 2025

Related U.S. Application Data
(63) Continuation-in-part of application No. PCT/US2023/073272, filed on Sep. 1, 2023.
(60) Provisional application No. 63/374,408, filed on Sep. 2, 2022.

(51) Int. Cl. A61K 35/76; A61K 39/00; A61K 39/12; A61P 31/14; C12N 15/86

(52) U.S. Cl. CPC A61K 35/76; A61K 39/12; A61P 31/14; C12N 15/86; A61K 2039/5256; A61K 2039/58

(57) ABSTRACT

The present invention relates to a vesicular stomatitis virus vaccine vector encoding a MARV glycoprotein (rVSVΔG-MARV-GP). Vaccination with as little as 200 plaque-forming units was 100% efficacious against MARV lethality and prevented development of viremia. rVSVΔG-MARV-GP vaccination induced MARV GP-specific serum IgG, and virus-neutralizing activity in serum was detectable in animals vaccinated with the highest doses.

### RELATED APPLICATIONS AND INCORPORATION BY REFERENCE

This application is a continuation-in-part of International Application No. PCT/US2023/073272 filed Sep. 1, 2023 and which published as International Publication No. WO 2024/050498 on Mar. 7, 2024 and which claims benefit of U.S. provisional patent application Ser. No. 63/374,408 filed Sep. 2, 2022.

The foregoing applications, and all documents cited therein or during their prosecution (“appln cited documents”) and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FEDERAL FUNDING LEGEND

This invention was made with government support under Grant No. MCDC-18-06-17-001 awarded by the Defense Threat Reduction Agency (DTRA) through the Medical CBRN Defense Consortium and Grant No. UC7AI094660 awarded by the Department of Health and Human Services, National Institutes of Health. The government has certain rights in the invention.

### SEQUENCE STATEMENT

The instant application contains a Sequence Listing which has been submitted via Patent Center and is hereby incorporated by reference in its entirety. Said .xml copy, created on Sep. 19, 2023 is named Y7969-02063 and is 17,472 bytes in size.

### FIELD OF THE INVENTION

The present invention relates to a vesicular stomatitis virus vaccine vector encoding a MARV glycoprotein (rVSVΔG-MARV-GP).

### BACKGROUND OF THE INVENTION

Filoviruses are a major threat to global health and continue to impact the health security and geopolitical stability of central and western Africa. The Filoviridae family is composed of the Ebolavirus and Marburgvirus genera. The Ebolavirus genus includes the species Zaire ebolavirus and six others while the Marburgvirus genus contains the single species Marburg marburgvirus [ictv.global/taxonomy]. A major Ebola virus (EBOV) outbreak occurred in West Africa in 2014-2016 and has been followed by a concerning frequency of outbreaks in the Democratic Republic of the Congo and Guinea [Sun, J., et al., Ann Med Surg (Lond), 2022. 79: p. 103958. Outbreaks have been caused by EBOV recrudescence-related events in non-endemic parts of West Africa as well as recent zoonotic transmission in endemic regions [Keita, A. K., et al., Nature, 2021. 597(7877): p. 539-543 and WHO. Ebola virus disease-Democratic Republic of the Congo. 2022 [cited 2022 Jul. 8]; Available from: www.who.int/emergencies/disease-outbreak-news/item/2022-DON377]. Other filoviruses remain endemic in animal reservoirs across Africa, including Marburg virus (MARV) and additional members of the Ebolavirus genus such as Sudan virus (SUDV) among others that cause lethal hemorrhagic fevers in humans and have similar epidemic potential to EBOV [Munster, V. J., et al., N Engl J Med, 2018. 379(13): p. 1198-1201]. Highlighting the risk of zoonotic transmission, modeling indicates that the geographic regions that might support transmission of MARV are quite extensive [Pigott, D. M., et al., Trans R Soc Trop Med Hyg, 2015. 109(6): p. 366-78]. Moreover, a transmission event was detected for the first time in West Africa in a patient from Guinea with no travel history [Koundouno et al., NEJM, 2022. 386(26): p. 2528-2530] and most recently in Ghana [WHO. Ghana reports first-ever suspected cases of Marburg virus disease. 2022 [cited 2022 Jul. 12]; Available from: www.afro.who.int/countries/ghana/news/ghana-reports-first-ever-suspected-cases-marburg-virus-disease]. Outbreaks of filoviruses including MARV will continue to happen at an accelerated rate in the future with factors such as climate change, increased inter-continental travel, population growth, and zoonotic reservoir range expansion contributing to the likelihood of future disease transmission events [Carlson, C. J., et al., Climate change increases cross-species viral transmission risk. Nature, 2022].

Vaccination against filoviruses in response to outbreaks and as a regular public health measure has the potential to help further control the health security threat to Africa. The success of the vaccine against EBOV produced by Merck Vaccines (rVSVΔG-ZEBOV-GP marketed as ERVEBO®) has provided a strong rationale for efforts to develop other recombinant, live-attenuated vaccines based on the vesicular stomatitis virus (VSV) vaccine vector technology [Tell, J. G., et al., Vaccines (Basel), 2020. 8(4) and Wolfe, D. N et al., Hum Vaccin Immunother, 2020. 16(11): p. 2855-2860]. The performance of ERVEBO in outbreak environments has clearly shown that the VSV-based technology has multiple features needed for development of other effective filovirus vaccines including, 1) acceptable safety and tolerability; 2) efficacy after a single dose; and 3) the ability to elicit protective immunity that develops rapidly [Tell, J. G., et al., Vaccines (Basel), 2020. 8(4), Wolfe, D. N et al., Hum Vaccin Immunother, 2020. 16(11): p. 2855-2860, Wolf, J., et al., Development of Pandemic Vaccines: ERVEBO Case Study. Vaccines (Basel), 2021. 9(3), Santoro, F., et al., Vaccines (Basel), 2021. 9(2) and Pinski, A. N. and I. Messaoudi, To B or Not to B: Mechanisms of Protection Conferred by rVSV-EBOV-GP and the Roles of Innate and Adaptive Immunity. Microorganisms, 2020. 8(10)].

In addition to the key rVSVΔG-ZEBOV-GP performance features mentioned above, it is also important to consider factors that affect access to filoviruses vaccines for populations where the viruses are endemic in Western and Central Africa. Availability of vaccine material that is safe for use in humans and can be deployed rapidly is important, as outbreaks of EBOV and other filoviruses such as MARV or SUDV cannot be forecasted with any certainty [Wolf, J., et al., Development of Pandemic Vaccines: ERVEBO Case Study. Vaccines (Basel), 2021]. Filovirus vaccines must also be cost-efficient, and thus dose-sparing conditions and efficacy following a single dose are important to evaluate. Finally, because of the sporadic nature of filovirus outbreaks, traditional human efficacy trials are not feasible. Thus, access to new filovirus vaccines will likely require use of existing alternative regulatory pathways such as the U.S. Food and Drug Administration (FDA) animal rule (https://www.fda.gov/emergency-preparedness-and-response/mcm-regulatory-science/animal-rule-information) and accelerated approval (https://www.fda.gov/drugs/information-health-care-professionals-drugs/accelerated-approval-program) as well as the generation of innovative data packages to demonstrate adequate safety, immunogenicity, and efficacy through preclinical animal studies and human clinical trials [Finch, C. L., et al., Vaccines (Basel), 2022. 10(3)]. In the case of VSV-based filovirus vaccines, this can be facilitated by the preclinical and clinical track record of rVSVΔG-ZEBOV-GP [Tell, J. G., et al., Vaccines (Basel), 2020. 8(4), Wolfe, D. N et al., Hum Vaccin Immunother, 2020. 16(11): p. 2855-2860, and Wolf, J., et al., Development of Pandemic Vaccines: ERVEBO Case Study. Vaccines (Basel), 2021. 9(3)] and the extensive preclinical research previously conducted on MARV and other filovirus vaccines based on the rVSVΔG-ZEBOV-GP design [Dulin, N., et al., Vaccine, 2021. 39(2): p. 202-208, Geisbert, T. W. and H. Feldmann, J Infect Dis, 2011. 204 Suppl 3: p. 51075-81 and Fathi, A. et al., Hum Vaccin Immunother, 2019. 15(10): p. 2269-2285].

In response to the first identified human MARV case in West Africa, the World Health Organization (WHO) convened a filovirus expert group comprised of infectious disease scientists, epidemiologists, public health experts, and vaccine developers, tasked to put together a research and development blueprint to enhance the WHO's “Strategic Agenda for Filoviruses Research and Monitoring” (AFIRM) [WHO. A WHO Strategic Agenda for Filovirus Research and Monitoring (AFIRM)-Roadmap Meeting. 2022 [cited 2022 Jul. 8]; Available from: www.who.int/news-room/events/detail/2022/03/30/default-calendar/save-the-date-a-who-strategic-agenda-for-filovirus-research-and-monitoring-(afirm)---roadmap-meeting]. Central to this blueprint is understanding what experimental MARV vaccines are available and their state of development, and what preclinical data is available that supports their use in an outbreak situation. Furthermore, the blueprint will cover development of clinical trial approaches that can be utilized during a public health emergency due to a MARV outbreak. It is likely that the use of ring vaccination, a strategy in which those most likely infected would receive immediate vaccination, could be implemented as early as possible in response to an emergence of MARV or other filovirus threats, and that the availability of clinical trial material, as was the case for Ebola Zaire, would aid in the response to future filovirus outbreaks [Dean, N. E. and I. M. Longini, Clin Trials, 2022: p. 17407745211073594]. Without the availability of vaccine prepared according to Good Manufacturing Practices (GMP) and ready for immediate use, the response to the 2014-2016 West African Ebola Zaire outbreak would have been much slower and had even more far-reaching consequences in terms of the toll on human lives and economically.

Citation or identification of any document in this application is not an admission that such document is available as prior art to the present invention.

### SUMMARY OF THE INVENTION

The present invention relates to recombinant vaccine which may comprise a nucleic acid sequence encoding a glycoprotein (GP) from the Musoke strain of Marburg virus (MARV) encoded in a VSV excluding a glycoprotein G gene (VSVΔG). In one embodiment, the nucleic acid may comprise a nucleic acid encoding an open reading frame of the MARV GP, such as SEQ ID NO: 1. In another embodiment, the nucleic acid may comprise nucleic acid encoding an open reading frame of the MARV GP in a VSVΔG such as SEQ ID NO: 2.

The present invention also relates to methods for vaccinating a mammal in need thereof with a vaccine which may comprise administering about 10²-10⁷ plaque-forming units (PFUs) of the vaccine to the mammal. The administering may be about 10⁴ PFU, about 10⁵ PFU or about 10⁶ PFU of the vaccine.

In one embodiment of the invention, the mammal may be a bat or a primate. The primate may be a bonobo, chimpanzee, gibbon, gorilla, monkey, orangutan or human. Advantageously, the primate is a human.

In another embodiment of the invention, the administration may be intramuscular (IM), intranasal (IN) or by an oral bait drop.

In another embodiment, the vaccine of the present invention may be administered as part of a combination vaccine, such as with an Ebola virus vaccine or a Sudan virus vaccine.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description, given by way of example, but not intended to limit the invention solely to the specific embodiments described, may best be understood in conjunction with the accompanying drawings.

FIG. 1A-1C. Schematic showing the design of the rVSVΔG-MARV-GP research vaccine.

FIG. 1A. Included at the top is a linear map of the MARV RNA genome illustrating that it encodes seven polypeptides (Abir, M. H., et al. (2022). “Pathogenicity and virulence of Marburg virus.” Virulence 13(1): 609-633). An illustration of the MARV virion is shown below the genome with the transmembrane GP incorporated in the membrane envelop and exposed on the surface of the particle.

FIG. 1B. The schematic shows the VSV RNA genome map, which encodes 5 structural proteins, with a virion shown below (Lyles, D. S., et al. (2013). Rhabdoviridae. Fields virology. D. M. Knipe and P. M. Howley. Philadelphia, Lippincott Williams and Wilkins. 1: 885-922). The research rVSVΔG-MARV-GP vaccine was developed by replacing the gene encoding VSV glycoprotein (G) with a gene that codes for MARV GP from the Musoke isolate (Garbutt, M., et al. (2004). “Properties of replication-competent vesicular stomatitis virus vectors expressing glycoproteins of filoviruses and arenaviruses.” J Virol 78(10): 5458-5465 and Jones, S. M., et al. (2005). “Live attenuated recombinant vaccine protects nonhuman primates against Ebola and Marburg viruses.” Nat Med 11(7): 786-790).

FIG. 1C. The VSVΔG-MARV-GP vaccine is a replication-competent chimeric virus that incorporates MARV GP on the surface of the virion.

FIG. 2. Summary of steps taken to regenerate the rVSVΔG-MARV-GP vaccine. RNA was extracted from a sample of the research vaccine expressing the GP from the MARV Musoke variant that was shown to be efficacious earlier (Garbutt, M., et al. (2004). “Properties of replication-competent vesicular stomatitis virus vectors expressing glycoproteins of filoviruses and arenaviruses.” J Virol 78(10): 5458-5465 and Jones, S. M., et al. (2005). “Live attenuated recombinant vaccine protects nonhuman primates against Ebola and Marburg viruses.” Nat Med 11(7): 786-790) and was used to determine the genomic nucleotide sequence. The GP gene nucleotide sequence was used subsequently to synthesize a new GP gene that was inserted into the VSV (Indiana serotype) genomic clone (Garbutt, M., et al. (2004). “Properties of replication-competent vesicular stomatitis virus vectors expressing glycoproteins of filoviruses and arenaviruses.” J Virol 78(10): 5458-5465; Schnell, M. J., et al. (1996). “The minimal conserved transcription stop-start signal promotes stable expression of a foreign gene in vesicular stomatitis virus.” J Virol 70(4): 2318-2323 and Lawson, N. D., et al. (1995). “Recombinant vesicular stomatitis viruses from DNA.” Proc Natl Acad Sci USA 92(10): 4477-4481) to generate a genomic plasmid DNA that could be used to regenerate a recombinant virus under conditions that would support development of a human vaccine.

FIG. 3A-3F. Generation and characterization of the rVSVΔG-MARV-GP vaccine for use in humans.

FIG. 3A. Schematic summarizing steps during rederivation of a rVSVΔG-MARV-GP chimeric virus suitable for human vaccine development. Recombinant virus was rederived from the genomic plasmid DNA (FIG. 2) after which 3 rounds virus plaque-isolation was conducted to develop clonal virus isolates. A pre-master virus seed (preMVS) subsequently was amplified and characterized to support good manufacturing practices (GMP) manufacturing. Virus derived from the preMVS was further evaluated by producing purified vaccine material that was used in a preclinical efficacy study.

FIG. 3B. Purified vaccine virus used in a preclinical efficacy study was analyzed by nanoflow cytometry or flow virometry to assess the uniformity of particles in the purified vaccine material [Ricci, G., et al., Sci Rep, 2021. 11(1): p. 7432].

FIG. 3C. Nanoflow cytometry or flow virometry also was used as an analysis tool during multiple stages of vaccine virus production as illustrated by analysis of virus produced during an independent vaccine production run. Nanoflow profiles are shown for multiple rVSVΔG-MARV-GP production stages including: harvested media (HM) from infected cultures of Vero cells, clarified harvest (CH), purification and concentrated using tangential flow filtration (TFF), following treatment with nuclease (post-benzonase treatment; PBT), post buffer exchange by TFF (BEP), and final purified product (FP).

FIG. 3D. Vaccine product characterization included analysis of MARV GP gene integrity by RT-PCR. A 2.9 Kb band is expected for an intact MARV-GP insert amplified by primers the bind in the VSV M and L genes as illustrated below the image of the agarose gel. Lanes: 1, 1Kb ladder; 2, the positive control VSVΔG-MARV-GP genomic plasmid; 3, a VSV genomic plasmid DNA in which the G gene was moved to the 5′ terminus of the genome so no transcription unit is present between M and L [Rabinovich, S., et al., PLoS One, 2014. 9(9): p. e106597]; 4, a no-template negative control; 5, HM; 6, no sample; 7, FP.

FIG. 3E. The Western blot procedure was used to analyze rVSVΔG-MARV-GP polypeptides from various vaccine production stages. VSV N (Rabinovich, S., et al. (2014). “A novel, live-attenuated vesicular stomatitis virus vector displaying conformationally intact, functional HIV-1 envelope trimers that elicits potent cellular and humoral responses in mice.” PLoS ONE 9(9): e106597) and MARV GP (IBT Bioservices) were detected using rabbit polyclonal antisera. The anti-GP antisera is specific for the GP2 subunit of MARV GP.

FIG. 3F. Expression of MARV GP and VSV N during infection of Vero cells. Vero cells infected with three different purified batches of rVSVΔG-MARV-GP were analyzed by flow cytometry to detect expression of MARV GP on the cell surface (monoclonal antibody 5C1; IBT Bioservices, Inc) and intracellular VSV N (monoclonal 10G4; Kerafast, Inc). Red is the profile of uninfected Vero cells and blue is infected cells.

FIG. 4A. Design of VSVΔG-MARV-GP preclinical dose-range efficacy study conducted in cynomolgus macaques. The top part of A illustrates the study schedule and table shows the study groups and VSVΔG-MARV-GP doses. The control VSVΔG-based Lassa virus vaccine (rVSVΔG-LASV-GPC; [Garbutt, M., et al., J Virol, 2004. 78(10): p. 5458-65 and Geisbert, T. W., et al., PLoS Med, 2005. 2(6): p. e183.]) was produced from an amplified virus (data not shown).

FIG. 4B. The graph shows survival after challenge with the MARV Angola isolate.

FIG. 4C. Plaque assays were conducted to assess viremia following MARV challenge. Infectious MARV was detected only in blood collected from control animals vaccinated with rVSVΔG-LASV-GPC.

FIG. 5. MARV RNA detected in blood after MARV Angola challenge. RNA was extracted from whole blood and was quantified by RT-qPCR. MARV genome copies in samples were calculated using a genome equivalent standard. The limit of detection was 1,000 copies/mL.

FIG. 6A-6B. Characterization of the antibody response induced by rVSVΔG-MARV-GP vaccination.

FIG. 6A. Enzyme-linked immunosorbent assay (ELISA) was performed using plates coated with a soluble form of MARV GP from the Angola isolate. Endpoint serum antibody titers are shown in the graph. Vaccine dose in PFUs is included at the right side of the graph. The lower detection limit is 100.

FIG. 6B. A plaque reduction assay based on serum neutralization of a rVSVΔG-MARV-GP (Musoke). Although similar, the VSV-based chimeric virus used for the neutralization assay was developed using a different VSV (Indiana) genomic clone and a GP (MARV Musoke) gene optimized using a VSV codon bias and procedures described earlier (Rabinovich, S., et al. (2014). “A novel, live-attenuated vesicular stomatitis virus vector displaying conformationally intact, functional HIV-1 envelope trimers that elicits potent cellular and humoral responses in mice.” PLoS ONE 9(9): e106597 and Espeseth, A. S., et al. (2022). “Preclinical immunogenicity and efficacy of a candidate COVID-19 vaccine based on a vesicular stomatitis virus-SARS-CoV-2 chimera.” EBioMedicine 82: 104203). The serum dilution at which rVSVΔG-MARV-GP plaque numbers were reduced by 50% (Neutralization titer 50 or NT 50) is plotted. The lower detection limit is 20.

### DETAILED DESCRIPTION OF THE INVENTION

A MARV vaccine candidate (rVSVΔG-MARV-GP) based on the recombinant VSV technology used for ERVEBO™ is being developed for use in people. A rVSVΔG-MARV-GP research vaccine has been shown to be safe and efficacious in multiple preclinical studies. To advance rVSVΔG-MARV-GP as a globally-accessible vaccine candidate for human use, Applicants regenerated a recombinant vaccine strain using conditions that would support future human vaccine development and tested it across a range of doses for immunogenicity and efficacy against MARV challenge in a cynomolgus macaque animal model for MARV disease. The rVSVΔG-MARV-GP vaccine was 100% efficacious against Marburg disease and protected against development of MARV viremia after a single IM injection even when doses as low as 200 PFUs were used. rVSVΔG-MARV-GP vaccination induced MARV GP-specific humoral responses that can be further interrogated to better understand correlates of protection and this data will provide an important bridge to future human safety and immunogenicity studies.

The present invention relates to a recombinant MARV vaccine encoding a MARV protein or a non-naturally occurring mutant thereof. Advantageously, the MARV protein is a MARV glycoprotein or a non-naturally occurring mutant thereof.

The Marburg virus is one of two members of the species Marburg marburgvirus, which is included in the genus Marburgvirus, family Filoviridae, and order Mononegavirales. Marburg virions consist of seven structural proteins. At the center is the helical ribonucleocapsid, which consists of the genomic RNA wrapped around a polymer of nucleoproteins (NP). Associated with the ribonucleoprotein is the RNA-dependent RNA polymerase (L) with the polymerase cofactor (VP35) and a transcription activator (VP30). The ribonucleoprotein is embedded in a matrix, formed by the major (VP40) and minor (VP24) matrix proteins. These particles are surrounded by a lipid membrane derived from the host cell membrane. The membrane anchors a glycoprotein (GP1,2) that projects 7 to 10 nm spikes away from its surface. Any of the structural proteins may be contemplated for a vaccine. Advantageously, the structural protein contemplated for a vaccine is the glycoprotein (GP).

Marburg virus (MARV) isolates include Angola, Musoke, and Ozolin. The Marburg viruses Musoke (MARV-Mus) and Angola (MARV-Ang) have highly similar genomic sequences. Advantageously, the strain is the MARV Musoke isolate.

The invention encompasses eliciting an immune response which may comprise systemically administering to an animal in need thereof an effective amount of any one of the non-naturally occurring protein(s) or any one of the nucleic acids encoding the non-naturally occurring protein(s) of the present invention, including nucleic acids that may have at least 80% or 85% or 90% or 95% homology or identity with a nucleotide encoding the sequence of the non-naturally occurring protein(s) of the invention. The animal may be a mammal, advantageously a primate, advantageously a human.

The invention pertains to the identification, design, synthesis and isolation of MARV proteins disclosed herein as well as nucleic acids encoding the same. The present invention also relates to homologues, derivatives and variants of the sequences of a MARV protein and nucleic acids encoding the same, wherein it is preferred that the homologue, derivative or variant have at least 50%, at least 60%, at least 70%, at least 75%, at least 80%, at least 85%, at least 90%, at least 93%, at least 95%, at least 97%, at least 98% or at least 99% homology or identity with the sequence of the MARV proteins or nucleic acids encoding the same. It is noted that within this specification, homology to sequences of the mutant proteins and nucleic acids encoding the same refers to the homology of the homologue, derivative or variant to the binding site of the mutant proteins and nucleic acids encoding the same.

The invention still further relates to nucleic acid sequences expressing the MARV proteins disclosed herein, or homologues, variants or derivatives thereof. One of skill in the art will know, recognize and understand techniques used to create such. Additionally, one of skill in the art will be able to incorporate such a nucleic acid sequence into an appropriate vector, allowing for production of the amino acid sequence of mutant proteins and nucleic acids encoding the same or a homologue, variant or derivative thereof.

Where used herein and unless specifically indicated otherwise, the following terms are intended to have the following meanings in addition to any broader (or narrower) meanings the terms might enjoy in the art:

The term “isolated” or “non-naturally occurring” is used herein to indicate that the isolated moiety (e.g. peptide or compound) exists in a physical milieu distinct from that in which it occurs in nature. For example, the isolated peptide may be substantially isolated with respect to the complex cellular milieu in which it naturally occurs. The absolute level of purity is not critical, and those skilled in the art may readily determine appropriate levels of purity according to the use to which the peptide is to be put. The term “isolating” when used a step in a process is to be interpreted accordingly.

In many circumstances, the isolated moiety will form part of a composition (for example a more or less crude extract containing many other molecules and substances), buffer system, matrix or excipient, which may for example contain other components (including proteins, such as albumin).

In other circumstances, the isolated moiety may be purified to essential homogeneity, for example as determined by PAGE or column chromatography (for example HPLC or mass spectrometry). In preferred embodiments, the isolated peptide or nucleic acid of the invention is essentially the sole peptide or nucleic acid in a given composition.

In an advantageous embodiment, a tag may be utilized for purification or biotinylation. The tag for purification may be a his tag. In another embodiment, the tag for biotinylation may be an avi-tag. Other tags are contemplated for purification, however, purification may be accomplished without a tag. In another embodiment, antibody (such as, not limited to, a broadly neutralizing antibody) affinity columns are contemplated. In another embodiment, lectin columns are contemplated.

The term “pharmaceutical composition” is used herein to define a solid or liquid composition in a form, concentration and level of purity suitable for administration to a patient (e.g. a human patient) upon which administration it may elicit the desired physiological changes. The terms “immunogenic composition” and “immunological composition” and “immunogenic or immunological composition” cover any composition that elicits an immune response against the targeted pathogen, Marburg viruses. Terms such as “vaccinal composition” and “vaccine” and “vaccine composition” cover any composition that induces a protective immune response against the targeted pathogen or which efficaciously protects against the pathogen; for instance, after administration or injection, elicits a protective immune response against the targeted pathogen or provides efficacious protection against the pathogen. Accordingly, an immunogenic or immunological composition induces an immune response, which may, but need not, be a protective immune response. An immunogenic or immunological composition may be used in the treatment of individuals infected with the pathogen, e.g., to stimulate an immune response against the pathogen, such as by stimulating antibodies against the pathogen. Thus, an immunogenic or immunological composition may be a pharmaceutical composition. Furthermore, when the text speaks of “immunogen, antigen or epitope”, an immunogen may be an antigen or an epitope of an antigen. A diagnostic composition is a composition containing a compound or antibody, e.g., a labeled compound or antibody, that is used for detecting the presence in a sample, such as a biological sample, e.g., blood, semen, vaginal fluid, etc., of an antibody that binds to the compound or an immunogen, antigen or epitope that binds to the antibody; for instance, an anti-MARV antibody or an MARV immunogen, antigen or epitope.

A “conservative amino acid change” is one in which the amino acid residue is replaced with an amino acid residue having a similar side chain. Families of amino acid residues having similar side chains have been defined in the art. These families include amino acids with basic side chains (e.g. lysine, arginine and histidine), acidic side chains (e.g. aspartic acid and glutamic acid), non-charged amino acids or polar side chains (e.g. glycine, asparagine, glutamine, serine, threonine, tyrosine and cysteine), non-polar side chains (e.g. alanine, valine, leucine, isoleucine, proline, phenylalanine, methionine and tryptophan), beta-branched side chains (e.g. threonine, valine and isoleucine), and aromatic side chains (e.g. tyrosine, phenylalanine, tryptophan and histidine).

The terms “protein”, “peptide”, “polypeptide”, and “amino acid sequence” are used interchangeably herein to refer to polymers of amino acid residues of any length. The polymer may be linear or branched, it may comprise modified amino acids or amino acid analogs, and it may be interrupted by chemical moieties other than amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling or bioactive component.

As used herein, the terms “antigen” or “immunogen” are used interchangeably to refer to a substance, typically a protein, which is capable of inducing an immune response in a subject. The term also refers to proteins that are immunologically active in the sense that once administered to a subject (either directly or by administering to the subject a nucleotide sequence or vector that encodes the protein) is able to evoke an immune response of the humoral and/or cellular type directed against that protein.

As used herein the terms “nucleotide sequences” and “nucleic acid sequences” refer to deoxyribonucleic acid (DNA) or ribonucleic acid (RNA) sequences, including, without limitation, messenger RNA (mRNA), DNA/RNA hybrids, or synthetic nucleic acids. The nucleic acid may be single-stranded, or partially or completely double-stranded (duplex). Duplex nucleic acids may be homoduplex or heteroduplex.

As used herein the term “transgene” may be used to refer to “recombinant” nucleotide sequences that may be derived from any of the nucleotide sequences encoding the proteins of the present invention. The term “recombinant” means a nucleotide sequence that has been manipulated “by man” and which does not occur in nature, or is linked to another nucleotide sequence or found in a different arrangement in nature. It is understood that manipulated “by man” means manipulated by some artificial means, including by use of machines, codon optimization, restriction enzymes, etc.

For example, in one embodiment the nucleotide sequences may be mutated such that the activity of the encoded proteins in vivo is abrogated. In another embodiment the nucleotide sequences may be codon optimized, for example the codons may be optimized for human use. In preferred embodiments the nucleotide sequences of the invention are both mutated to abrogate the normal in vivo function of the encoded proteins, and codon optimized for human use. For example, each of the sequences of the invention, such as the MARV proteins, may be altered in these ways.

As regards codon optimization, the nucleic acid molecules of the invention have a nucleotide sequence that encodes the antigens of the invention and may be designed to employ codons that are used in the genes of the subject in which the antigen is to be produced. Many viruses use a large number of rare codons and, by altering these codons to correspond to codons commonly used in the desired subject, enhanced expression of the antigens may be achieved. In a preferred embodiment, the codons used are “humanized” codons, i.e., the codons are those that appear frequently in highly expressed human genes (Andre et al., J. Virol. 72:1497-1503, 1998) instead of those codons that are frequently used by MARV. Such codon usage provides for efficient expression of the transgenic MARV proteins in human cells. Any suitable method of codon optimization may be used. Such methods, and the selection of such methods, are well known to those of skill in the art. In addition, there are several companies that will optimize codons of sequences, such as Geneart (geneart.com). Thus, the nucleotide sequences of the invention may readily be codon optimized.

The invention further encompasses nucleotide sequences encoding functionally and/or antigenically equivalent variants and derivatives of the antigens of the invention and functionally equivalent fragments thereof. These functionally equivalent variants, derivatives, and fragments display the ability to retain antigenic activity. For instance, changes in a DNA sequence that do not change the encoded amino acid sequence, as well as those that result in conservative substitutions of amino acid residues, one or a few amino acid deletions or additions, and substitution of amino acid residues by amino acid analogs are those which will not significantly affect properties of the encoded polypeptide. Conservative amino acid substitutions are glycine/alanine; valine/isoleucine/leucine; asparagine/glutamine; aspartic acid/glutamic acid; serine/threonine/methionine; lysine/arginine; and phenylalanine/tyrosine/tryptophan. In one embodiment, the variants have at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% homology or identity to the antigen, epitope, immunogen, peptide or polypeptide or a nucleotide sequence encoding the same of interest.

For the purposes of the present invention, sequence identity or homology is determined by comparing the sequences when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin & Altschul, Proc. Natl. Acad. Sci. USA 1990; 87: 2264-2268, modified as in Karlin & Altschul, Proc. Natl. Acad. Sci. USA 1993; 90: 5873-5877.

Another example of a mathematical algorithm used for comparison of sequences is the algorithm of Myers & Miller, CABIOS 1988; 4: 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 may be used. Yet another useful algorithm for identifying regions of local sequence similarity and alignment is the FASTA algorithm as described in Pearson & Lipman, Proc. Natl. Acad. Sci. USA 1988; 85: 2444-2448.

Advantageous for use according to the present invention is the WU-BLAST (Washington University BLAST) version 2.0 software. WU-BLAST version 2.0 executable programs for several UNIX platforms may be downloaded from ftp://blast.wustl.edu/blast/executables. This program is based on WU-BLAST version 1.4, which in turn is based on the public domain NCBI-BLAST version 1.4 (Altschul & Gish, 1996, Local alignment statistics, Doolittle ed., Methods in Enzymology 266: 460-480; Altschul et al., Journal of Molecular Biology 1990; 215: 403-410; Gish & States, 1993; Nature Genetics 3: 266-272; Karlin & Altschul, 1993; Proc. Natl. Acad. Sci. USA 90: 5873-5877; all of which are incorporated by reference herein).

The various recombinant nucleotide sequences and immunogens of the invention are made using standard recombinant DNA and cloning techniques. Such techniques are well known to those of skill in the art. See for example, “Molecular Cloning: A Laboratory Manual”, second edition (Sambrook et al. 1989).

The nucleotide sequences of the present invention may be inserted into “vectors.” The term “vector” is widely used and understood by those of skill in the art, and as used herein the term “vector” is used consistent with its meaning to those of skill in the art. For example, the term “vector” is commonly used by those skilled in the art to refer to a vehicle that allows or facilitates the transfer of nucleic acid molecules from one environment to another or that allows or facilitates the manipulation of a nucleic acid molecule.

Any vector that allows expression of the immunogen of the present invention may be used in accordance with the present invention. In certain embodiments, the immunogen of the present invention may be used in vitro (such as using cell-free expression systems) and/or in cultured cells grown in vitro in order to produce the encoded immunogens, which may then be used for various applications such as in the production of proteinaceous vaccines. For such applications, any vector that allows expression of the immunogens in vitro and/or in cultured cells may be used.

For applications where it is desired that the immunogens be expressed in vivo, for example when the transgenes of the invention are used in DNA or DNA-containing vaccines, any vector that allows for the expression of the antibodies of the present invention and is safe for use in vivo may be used. In preferred embodiments the vectors used are safe for use in humans, mammals and/or laboratory animals.

For the immunogens of the present invention to be expressed, the protein coding sequence should be “operably linked” to regulatory or nucleic acid control sequences that direct transcription and translation of the protein. As used herein, a coding sequence and a nucleic acid control sequence or promoter are said to be “operably linked” when they are covalently linked in such a way as to place the expression or transcription and/or translation of the coding sequence under the influence or control of the nucleic acid control sequence. The “nucleic acid control sequence” may be any nucleic acid element, such as, but not limited to promoters, enhancers, internal ribosome entry site (IRES), introns, and other elements described herein that direct the expression of a nucleic acid sequence or coding sequence that is operably linked thereto. For VSV, the gene also can be operably linked to intergenic regions that control gene expression.

The vectors used in accordance with the present invention should typically be chosen such that they contain a suitable gene regulatory region, such as a promoter or intergenic region, such that the immunogen of the invention may be expressed.

Any suitable vector may be used depending on the application. For example, plasmids, viral vectors, bacterial vectors, protozoal vectors, insect vectors, baculovirus expression vectors, yeast vectors, mammalian cell vectors, and the like, may be used. Suitable vectors may be selected by the skilled artisan taking into consideration the characteristics of the vector and the requirements for expressing the immunogens under the identified circumstances.

In preferred embodiments of the present invention viral vectors are used. Viral expression vectors are well known to those skilled in the art and include, for example, viruses such as adenoviruses, adeno-associated viruses (AAV), alphaviruses, herpesviruses, retroviruses and poxviruses, including avipox viruses, attenuated poxviruses, vaccinia viruses, and particularly, the modified vaccinia Ankara virus (MVA; ATCC Accession No. VR-1566). Such viruses, when used as expression vectors are innately non-pathogenic in the selected subjects such as humans or have been modified to render them non-pathogenic in the selected subjects. For example, replication-defective adenoviruses and alphaviruses are well known and may be used as gene delivery vectors.

Advantageously, the vector is a vesicular stomatitis virus (VSV) vector.

VSV is a very practical, safe, and immunogenic vector for conducting animal studies, and an attractive candidate for developing vaccines for use in humans, as shown by development of a marketed Ebola virus vaccine (ERVEBO®). VSV is a member of the Rhabdoviridae family of enveloped viruses containing a nonsegmented, negative-sense RNA genome. The genome is composed of 5 genes arranged sequentially 3′-N-P-M-G-L-5′, each encoding a polypeptide found in mature virions. Notably, the surface glycoprotein G is a transmembrane polypeptide that is present in the viral envelope as a homotrimer, and like MARV GP, it mediates cell attachment and infection.

Advantageously, the VSV vector is replication deficient due to a deletion of the glycoprotein G gene (VSVΔG). In one embodiment, the VSV G gene is replaced by a gene encoding a MARV protein or fragments thereof. In a second embodiment, VSV G is a carrier or scaffold for MARV epitopes. The disclosures of U.S. Pat. Nos. 9,610,346, 9,802,986 and 10,844,095 are incorporated by reference. In another embodiment, the MARV GP replacing G is functional.

The VSV vector may be replication deficient due to a deletion of the glycoprotein G gene (VSVΔG). In one embodiment, the VSV G is replaced by a gene encoding MARV protein or fragments thereof. The disclosures of U.S. Pat. Nos. 9,610,346, 9,802,986 and 10,844,095 are incorporated by reference. The VSVΔG vector of Espeseth et al. (eBioMedicine 2022; 00:104203 Published online at doi.org/10.1016/j.ebiom.2022.104203) is also contemplated.

In one embodiment, a viral genome sequence for the preMVS comprises a coding sequence of

(SEQ ID NO: 1)
ATGAAGACCACATGTTTCCTTATCAGTCTTATCTTAATTCAAGGGACAAAAAATCTC
CCCATTTTAGAGATAGCTAGTAATAATCAACCCCAAAATGTGGATTCGGTATGCTCC
GGAACTCTCCAGAAGACAGAAGACGTCCATCTGATGGGATTCACACTGAGTGGGCA
AAAAGTTGCTGATTCCCCTTTGGAGGCATCCAAGCGATGGGCTTTCAGGACAGGTGT
ACCTCCCAAGAATGTTGAGTACACAGAGGGGGAGGAAGCCAAAACATGCTACAATA
TAAGTGTAACGGATCCCTCTGGAAAATCCTTGCTGTTAGATCCTCCTACCAACATCC
GTGACTATCCTAAATGCAAAACTATCCATCATATTCAAGGTCAAAACCCTCATGCAC
AGGGGATCGCCCTTCATTTATGGGGAGCATTTTTTCTGTATGATCGCATTGCCTCCAC
AACAATGTACCGAGGCAAAGTCTTCACTGAAGGGAACATAGCAGCTATGATTGTCA
ATAAGACAGTGCACAAAATGATTTTCTCGCGGCAAGGACAAGGGTACCGTCATATG
AATCTGACTTCTACTAATAAATATTGGACAAGTAGTAACGGAACGCAAACGAATGA
CACTGGATGTTTCGGCGCTCTTCAAGAATACAATTCTACAAAGAACCAAACATGTGC
TCCGTCCAAAATACCTCCACCACTGCCCACAGCCCGTCCGGAGATCAAACTCACAAG
CACCCCAACTGATGCCACCAAACTCAATACCACGGACCCAAGCAGTGATGATGAGG
ACCTCGCAACATCCGGCTCAGGGTCCGGAGAACGAGAACCCCACACAACTTCTGAT
GCGGTCACCAAGCAAGGGCTTTCATCAACAATGCCACCCACTCCCTCACCACAACCA
AGCACGCCACAGCAAGGAGGAAACAACACAAACCATTCCCAAGATGCTGTGACTGA
ACTAGACAAAAATAACACAACTGCACAACCGTCCATGCCCCCTCATAACACTACCA
CAATCTCTACTAACAACACCTCCAAACACAACTTCAGCACTCTCTCTGCACCATTAC
AAAACACCACCAATGACAACACACAGAGCACAATCACTGAAAATGAGCAAACCAG
TGCCCCCTCGATAACAACCCTGCCTCCAACGGGAAATCCCACCACAGCAAAGAGCA
CCAGCAGCAAAAAAGGCCCCGCCACAACGGCACCAAACACGACAAATGAGCATTTC
ACCAGTCCTCCCCCCACCCCCAGCTCGACTGCACAACATCTTGTATATTTCAGAAGA
AAGCGAAGTATCCTCTGGAGGGAAGGCGACATGTTCCCTTTTCTGGATGGGTTAATA
AATGCTCCAATTGATTTTGACCCAGTTCCAAATACAAAAACAATCTTTGATGAATCC
TCTAGTTCTGGTGCCTCGGCTGAGGAAGATCAACATGCCTCCCCCAATATTAGTTTA
ACTTTATCTTATTTTCCTAATATAAATGAGAACACTGCCTACTCTGGAGAAAATGAG
AATGATTGTGATGCAGAGTTAAGAATTTGGAGCGTTCAGGAGGATGACCTGGCCGC
AGGGCTCAGTTGGATACCGTTTTTTGGCCCTGGAATTGAAGGACTTTACACTGCTGT
TTTAATTAAAAATCAAAACAATTTGGTCTGCAGGTTGAGGCGTCTAGCCAATCAAAC
TGCCAAATCCTTGGAACTCTTATTGAGAGTCACAACTGAGGAAAGAACATTCTCCTT
AATCAATAGACATGCTATTGACTTTCTACTCACAAGATGGGGAGGAACATGCAAAG
TGCTTGGACCTGATTGTTGCATCGGGATAGAAGACTTGTCCAAAAATATTTCAGAGC
AAATTGACCAAATTAAAAAGGACGAACAAAAAGAGGGGACTGGTTGGGGTCTGGGT
GGTAAATGGTGGACATCCGACTGGGGTGTTCTTACTAACTTGGGCATTTTGCTACTA
TTATCCATAGCTGTCTTGATTGCTCTATCCTGTATTTGTCGTATCTTTACTAAATATAT
CGGATAA.

In a particularly advantageous embodiment, the vector is a rVSVΔG-MARV-GP vector. In a particularly advantageous embodiment, a viral genome sequence for the preMVS comprises

(SEQ ID NO: 2)
ACGAAGACAAACAAACCATTATTATCATTAAAAGGCTCAGGAGAAACTTTAACAGT
AATCAAAATGTCTGTTACAGTCAAGAGAATCATTGACAACACAGTCGTAGTTCCAAA
ACTTCCTGCAAATGAGGATCCAGTGGAATACCCGGCAGATTACTTCAGAAAATCAA
AGGAGATTCCTCTTTACATCAATACTACAAAAAGTTTGTCAGATCTAAGAGGATATG
TCTACCAAGGCCTCAAATCCGGAAATGTATCAATCATACATGTCAACAGCTACTTGT
ATGGAGCATTAAAGGACATCCGGGGTAAGTTGGATAAAGATTGGTCAAGTTTCGGA
ATAAACATCGGGAAAGCAGGGGATACAATCGGAATATTTGACCTTGTATCCTTGAA
AGCCCTGGACGGCGTACTTCCAGATGGAGTATCGGATGCTTCCAGAACCAGCGCAG
ATGACAAATGGTTGCCTTTGTATCTACTTGGCTTATACAGAGTGGGCAGAACACAAA
TGCCTGAATACAGAAAAAAGCTCATGGATGGGCTGACAAATCAATGCAAAATGATC
AATGAACAGTTTGAACCTCTTGTGCCAGAAGGTCGTGACATTTTTGATGTGTGGGGA
AATGACAGTAATTACACAAAAATTGTCGCTGCAGTGGACATGTTCTTCCACATGTTC
AAAAAACATGAATGTGCCTCGTTCAGATACGGAACTATTGTTTCCAGATTCAAAGAT
TGTGCTGCATTGGCAACATTTGGACACCTCTGCAAAATAACCGGAATGTCTACAGAA
GATGTAACGACCTGGATCTTGAACCGAGAAGTTGCAGATGAAATGGTCCAAATGAT
GCTTCCAGGCCAAGAAATTGACAAGGCCGATTCATACATGCCTTATTTGATCGACTT
TGGATTGTCTTCTAAGTCTCCATATTCTTCCGTCAAAAACCCTGCCTTCCACTTCTGG
GGGCAATTGACAGCTCTTCTGCTCAGATCCACCAGAGCAAGGAATGCCCGACAGCC
TGATGACATTGAGTATACATCTCTTACTACAGCAGGTTTGTTGTACGCTTATGCAGTA
GGATCCTCTGCCGACTTGGCACAACAGTTTTGTGTTGGAGATAACAAATACACTCCA
GATGATAGTACCGGAGGATTGACGACTAATGCACCGCCACAAGGCAGAGATGTGGT
CGAATGGCTCGGATGGTTTGAAGATCAAAACAGAAAACCGACTCCTGATATGATGC
AGTATGCGAAAAGAGCAGTCATGTCACTGCAAGGCCTAAGAGAGAAGACAATTGGC
AAGTATGCTAAGTCAGAATTTGACAAATGACCCTATAATTCTCAGATCACCTATTAT
ATATTATGCTACATATGAAAAAAACTAACAGATATCATGGATAATCTCACAAAAGTT
CGTGAGTATCTCAAGTCCTATTCTCGTCTGGATCAGGCGGTAGGAGAGATAGATGAG
ATCGAAGCACAACGAGCTGAAAAGTCCAATTATGAGTTGTTCCAAGAGGATGGAGT
GGAAGAGCATACTAAGCCCTCTTATTTTCAGGCAGCAGATGATTCTGACACAGAATC
TGAACCAGAAATTGAAGACAATCAAGGCTTGTATGCACCAGATCCAGAAGCTGAGC
AAGTTGAAGGCTTTATACAGGGGCCTTTAGATGACTATGCAGATGAGGAAGTGGAT
GTTGTATTTACTTCGGACTGGAAACAGCCTGAGCTTGAATCTGACGAGCATGGAAAG
ACCTTACGGTTGACATCGCCAGAGGGTTTAAGTGGAGAGCAGAAATCCCAGTGGCT
TTCGACGATTAAAGCAGTCGTGCAAAGTGCCAAATACTGGAATCTGGCAGAGTGCA
CATTTGAAGCATCGGGAGAAGGGGTCATTATGAAGGAGCGCCAGATAACTCCGGAT
GTATATAAGGTCACTCCAGTGATGAACACACATCCGTCCCAATCAGAAGCAGTATCA
GATGTTTGGTCTCTCTCAAAGACATCCATGACTTTCCAACCCAAGAAAGCAAGTCTT
CAGCCTCTCACCATATCCTTGGATGAATTGTTCTCATCTAGAGGAGAGTTCATCTCTG
TCGGAGGTGACGGACGAATGTCTCATAAAGAGGCCATCCTGCTCGGCCTGAGATAC
AAAAAGTTGTACAATCAGGCGAGAGTCAAATATTCTCTGTAGACTATGAAAAAAAG
TAACAGATATCACGATCTAAGTGTTATCCCAATCCATTCATCATGAGTTCCTTAAAG
AAGATTCTCGGTCTGAAGGGGAAAGGTAAGAAATCTAAGAAATTAGGGATCGCACC
ACCCCCTTATGAAGAGGACACTAGCATGGAGTATGCTCCGAGCGCTCCAATTGACA
AATCCTATTTTGGAGTTGACGAGATGGACACCTATGATCCGAATCAATTAAGATATG
AGAAATTCTTCTTTACAGTGAAAATGACGGTTAGATCTAATCGTCCGTTCAGAACAT
ACTCAGATGTGGCAGCCGCTGTATCCCATTGGGATCACATGTACATCGGAATGGCAG
GGAAACGTCCCTTCTACAAAATCTTGGCTTTTTTGGGTTCTTCTAATCTAAAGGCCAC
TCCAGCGGTATTGGCAGATCAAGGTCAACCAGAGTATCACGCTCACTGCGAAGGCA
GGGCTTATTTGCCACATAGGATGGGGAAGACCCCTCCCATGCTCAATGTACCAGAGC
ACTTCAGAAGACCATTCAATATAGGTCTTTACAAGGGAACGATTGAGCTCACAATGA
CCATCTACGATGATGAGTCACTGGAAGCAGCTCCTATGATCTGGGATCATTTCAATT
CTTCCAAATTTTCTGATTTCAGAGAGAAGGCCTTAATGTTTGGCCTGATTGTCGAGA
AAAAGGCATCTGGAGCGTGGGTCCTGGACTCTATCGGCCACTTCAAATGAGCTAGTC
TAACTTCTAGCTTCTGAACAATCCCCGGTTTACTCAGTCTCCCCTAATTCCAGCCTCT
CGAACAACTAATATCCTGTCTTTTCTATCCCTATGAAAAAAACTAACAGAGATCGAT
CTGTTTACGCGCTAGTGGATCCTACTCGAGAACATGAAGACCACATGTTTCCTTATC
AGTCTTATCTTAATTCAAGGGACAAAAAATCTCCCCATTTTAGAGATAGCTAGTAAT
AATCAACCCCAAAATGTGGATTCGGTATGCTCCGGAACTCTCCAGAAGACAGAAGA
CGTCCATCTGATGGGATTCACACTGAGTGGGCAAAAAGTTGCTGATTCCCCTTTGGA
GGCATCCAAGCGATGGGCTTTCAGGACAGGTGTACCTCCCAAGAATGTTGAGTACA
CAGAGGGGGAGGAAGCCAAAACATGCTACAATATAAGTGTAACGGATCCCTCTGGA
AAATCCTTGCTGTTAGATCCTCCTACCAACATCCGTGACTATCCTAAATGCAAAACT
ATCCATCATATTCAAGGTCAAAACCCTCATGCACAGGGGATCGCCCTTCATTTATGG
GGAGCATTTTTTCTGTATGATCGCATTGCCTCCACAACAATGTACCGAGGCAAAGTC
TTCACTGAAGGGAACATAGCAGCTATGATTGTCAATAAGACAGTGCACAAAATGAT
TTTCTCGCGGCAAGGACAAGGGTACCGTCATATGAATCTGACTTCTACTAATAAATA
TTGGACAAGTAGTAACGGAACGCAAACGAATGACACTGGATGTTTCGGCGCTCTTC
AAGAATACAATTCTACAAAGAACCAAACATGTGCTCCGTCCAAAATACCTCCACCA
CTGCCCACAGCCCGTCCGGAGATCAAACTCACAAGCACCCCAACTGATGCCACCAA
ACTCAATACCACGGACCCAAGCAGTGATGATGAGGACCTCGCAACATCCGGCTCAG
GGTCCGGAGAACGAGAACCCCACACAACTTCTGATGCGGTCACCAAGCAAGGGCTT
TCATCAACAATGCCACCCACTCCCTCACCACAACCAAGCACGCCACAGCAAGGAGG
AAACAACACAAACCATTCCCAAGATGCTGTGACTGAACTAGACAAAAATAACACAA
CTGCACAACCGTCCATGCCCCCTCATAACACTACCACAATCTCTACTAACAACACCT
CCAAACACAACTTCAGCACTCTCTCTGCACCATTACAAAACACCACCAATGACAACA
CACAGAGCACAATCACTGAAAATGAGCAAACCAGTGCCCCCTCGATAACAACCCTG
CCTCCAACGGGAAATCCCACCACAGCAAAGAGCACCAGCAGCAAAAAAGGCCCCG
CCACAACGGCACCAAACACGACAAATGAGCATTTCACCAGTCCTCCCCCCACCCCC
AGCTCGACTGCACAACATCTTGTATATTTCAGAAGAAAGCGAAGTATCCTCTGGAGG
GAAGGCGACATGTTCCCTTTTCTGGATGGGTTAATAAATGCTCCAATTGATTTTGAC
CCAGTTCCAAATACAAAAACAATCTTTGATGAATCCTCTAGTTCTGGTGCCTCGGCT
GAGGAAGATCAACATGCCTCCCCCAATATTAGTTTAACTTTATCTTATTTTCCTAATA
TAAATGAGAACACTGCCTACTCTGGAGAAAATGAGAATGATTGTGATGCAGAGTTA
AGAATTTGGAGCGTTCAGGAGGATGACCTGGCCGCAGGGCTCAGTTGGATACCGTTT
TTTGGCCCTGGAATTGAAGGACTTTACACTGCTGTTTTAATTAAAAATCAAAACAAT
TTGGTCTGCAGGTTGAGGCGTCTAGCCAATCAAACTGCCAAATCCTTGGAACTCTTA
TTGAGAGTCACAACTGAGGAAAGAACATTCTCCTTAATCAATAGACATGCTATTGAC
TTTCTACTCACAAGATGGGGAGGAACATGCAAAGTGCTTGGACCTGATTGTTGCATC
GGGATAGAAGACTTGTCCAAAAATATTTCAGAGCAAATTGACCAAATTAAAAAGGA
CGAACAAAAAGAGGGGACTGGTTGGGGTCTGGGTGGTAAATGGTGGACATCCGACT
GGGGTGTTCTTACTAACTTGGGCATTTTGCTACTATTATCCATAGCTGTCTTGATTGC
TCTATCCTGTATTTGTCGTATCTTTACTAAATATATCGGATAATAAGCTAGCTGTTTA
CGCGTTATCCATGCTCAAAGAGGCCTCAATTATATTTGAGTTTTTAATTTTTATGAAA
AAAACTAACAGCAATCATGGAAGTCCACGATTTTGAGACCGACGAGTTCAATGATTT
CAATGAAGATGACTATGCCACAAGAGAATTCCTGAATCCCGATGAGCGCATGACGT
ACTTGAATCATGCTGATTACAACCTGAATTCTCCTCTAATTAGTGATGATATTGACA
ATTTAATCAGGAAATTCAATTCTCTTCCAATTCCCTCGATGTGGGATAGTAAGAACT
GGGATGGAGTTCTTGAGATGTTAACGTCATGTCAAGCCAATCCCATCCCAACATCTC
AGATGCATAAATGGATGGGAAGTTGGTTAATGTCTGATAATCATGATGCCAGTCAA
GGGTATAGTTTTTTACATGAAGTGGACAAAGAGGCAGAAATAACATTTGACGTGGT
GGAGACCTTCATCCGCGGCTGGGGCAACAAACCAATTGAATACATCAAAAAGGAAA
GATGGACTGACTCATTCAAAATTCTCGCTTATTTGTGTCAAAAGTTTTTGGACTTACA
CAAGTTGACATTAATCTTAAATGCTGTCTCTGAGGTGGAATTGCTCAACTTGGCGAG
GACTTTCAAAGGCAAAGTCAGAAGAAGTTCTCATGGAACGAACATATGCAGGATTA
GGGTTCCCAGCTTGGGTCCTACTTTTATTTCAGAAGGATGGGCTTACTTCAAGAAAC
TTGATATTCTAATGGACCGAAACTTTCTGTTAATGGTCAAAGATGTGATTATAGGGA
GGATGCAAACGGTGCTATCCATGGTATGTAGAATAGACAACCTGTTCTCAGAGCAA
GACATCTTCTCCCTTCTAAATATCTACAGAATTGGAGATAAAATTGTGGAGAGGCAG
GGAAATTTTTCTTATGACTTGATTAAAATGGTGGAACCGATATGCAACTTGAAGCTG
ATGAAATTAGCAAGAGAATCAAGGCCTTTAGTCCCACAATTCCCTCATTTTGAAAAT
CATATCAAGACTTCTGTTGATGAAGGGGCAAAAATTGACCGAGGTATAAGATTCCTC
CATGATCAGATAATGAGTGTGAAAACAGTGGATCTCACACTGGTGATTTATGGATCG
TTCAGACATTGGGGTCATCCTTTTATAGATTATTACACTGGACTAGAAAAATTACAT
TCCCAAGTAACCATGAAGAAAGATATTGATGTGTCATATGCAAAAGCACTTGCAAG
TGATTTAGCTCGGATTGTTCTATTTCAACAGTTCAATGATCATAAAAAGTGGTTCGTG
AATGGAGACTTGCTCCCTCATGATCATCCCTTTAAAAGTCATGTTAAAGAAAATACA
TGGCCCACAGCTGCTCAAGTTCAAGATTTTGGAGATAAATGGCATGAACTTCCGCTG
ATTAAATGTTTTGAAATACCCGACTTACTAGACCCATCGATAATATACTCTGACAAA
AGTCATTCAATGAATAGGTCAGAGGTGTTGAAACATGTCCGAATGAATCCGAACAC
TCCTATCCCTAGTAAAAAGGTGTTGCAGACTATGTTGGACACAAAGGCTACCAATTG
GAAAGAATTTCTTAAAGAGATTGATGAGAAGGGCTTAGATGATGATGATCTAATTAT
TGGTCTTAAAGGAAAGGAGAGGGAACTGAAGTTGGCAGGTAGATTTTTCTCCCTAAT
GTCTTGGAAATTGCGAGAATACTTTGTAATTACCGAATATTTGATAAAGACTCATTT
CGTCCCTATGTTTAAAGGCCTGACAATGGCGGACGATCTAACTGCAGTCATTAAAAA
GATGTTAGATTCCTCATCCGGCCAAGGATTGAAGTCATATGAGGCAATTTGCATAGC
CAATCACATTGATTACGAAAAATGGAATAACCACCAAAGGAAGTTATCAAACGGCC
CAGTGTTCCGAGTTATGGGCCAGTTCTTAGGTTATCCATCCTTAATCGAGAGAACTC
ATGAATTTTTTGAGAAAAGTCTTATATACTACAATGGAAGACCAGACTTGATGCGTG
TTCACAACAACACACTGATCAATTCAACCTCCCAACGAGTTTGTTGGCAAGGACAAG
AGGGTGGACTGGAAGGTCTACGGCAAAAAGGATGGAGTATCCTCAATCTACTGGTT
ATTCAAAGAGAGGCTAAAATCAGAAACACTGCTGTCAAAGTCTTGGCACAAGGTGA
TAATCAAGTTATTTGCACACAGTATAAAACGAAGAAATCGAGAAACGTTGTAGAAT
TACAGGGTGCTCTCAATCAAATGGTTTCTAATAATGAGAAAATTATGACTGCAATCA
AAATAGGGACAGGGAAGTTAGGACTTTTGATAAATGACGATGAGACTATGCAATCT
GCAGATTACTTGAATTATGGAAAAATACCGATTTTCCGTGGAGTGATTAGAGGGTTA
GAGACCAAGAGATGGTCACGAGTGACTTGTGTCACCAATGACCAAATACCCACTTG
TGCTAATATAATGAGCTCAGTTTCCACAAATGCTCTCACCGTAGCTCATTTTGCTGAG
AACCCAATCAATGCCATGATACAGTACAATTATTTTGGGACATTTGCTAGACTCTTG
TTGATGATGCATGATCCTGCTCTTCGTCAATCATTGTATGAAGTTCAAGATAAGATA
CCAGGCTTGCACAGTTCTACTTTCAAATACGCCATGTTGTATTTGGACCCTTCCATTG
GAGGAGTGTCGGGCATGTCTTTGTCCAGGTTTTTGATTAGAGCCTTCCCAGATCCCG
TAACAGAAAGTCTCTCATTCTGGAGATTCATCCATGTACATGCTCGAAGTGAGCATC
TGAAGGAGATGAGTGCAGTATTTGGAAACCCCGAGATAGCCAAGTTTCGAATAACT
CACATAGACAAGCTAGTAGAAGATCCAACCTCTCTGAACATCGCTATGGGAATGAG
TCCAGCGAACTTGTTAAAGACTGAGGTTAAAAAATGCTTAATCGAATCAAGACAAA
CCATCAGGAACCAGGTGATTAAGGATGCAACCATATATTTGTATCATGAAGAGGAT
CGGCTCAGAAGTTTCTTATGGTCAATAAATCCTCTGTTCCCTAGATTTTTAAGTGAAT
TCAAATCAGGCACTTTTTTGGGAGTCGCAGACGGGCTCATCAGTCTATTTCAAAATT
CTCGTACTATTCGGAACTCCTTTAAGAAAAAGTATCATAGGGAATTGGATGATTTGA
TTGTGAGGAGTGAGGTATCCTCTTTGACACATTTAGGGAAACTTCATTTGAGAAGGG
GATCATGTAAAATGTGGACATGTTCAGCTACTCATGCTGACACATTAAGATACAAAT
CCTGGGGCCGTACAGTTATTGGGACAACTGTACCCCATCCATTAGAAATGTTGGGTC
CACAACATCGAAAAGAGACTCCTTGTGCACCATGTAACACATCAGGGTTCAATTATG
TTTCTGTGCATTGTCCAGACGGGATCCATGACGTCTTTAGTTCACGGGGACCATTGC
CTGCTTATCTAGGGTCTAAAACATCTGAATCTACATCTATTTTGCAGCCTTGGGAAA
GGGAAAGCAAAGTCCCACTGATTAAAAGAGCTACACGTCTTAGAGATGCTATCTCTT
GGTTTGTTGAACCCGACTCTAAACTAGCAATGACTATACTTTCTAACATCCACTCTTT
AACAGGCGAAGAATGGACCAAAAGGCAGCATGGGTTCAAAAGAACAGGGTCTGCC
CTTCATAGGTTTTCGACATCTCGGATGAGCCATGGTGGGTTCGCATCTCAGAGCACT
GCAGCATTGACCAGGTTGATGGCAACTACAGACACCATGAGGGATCTGGGAGATCA
GAATTTCGACTTTTTATTCCAAGCAACGTTGCTCTATGCTCAAATTACCACCACTGTT
GCAAGAGACGGATGGATCACCAGTTGTACAGATCATTATCATATTGCCTGTAAGTCC
TGTTTGAGACCCATAGAAGAGATCACCCTGGACTCAAGTATGGACTACACGCCCCC
AGATGTATCCCATGTGCTGAAGACATGGAGGAATGGGGAAGGTTCGTGGGGACAAG
AGATAAAACAGATCTATCCTTTAGAAGGGAATTGGAAGAATTTAGCACCTGCTGAG
CAATCCTATCAAGTCGGCAGATGTATAGGTTTTCTATATGGAGACTTGGCGTATAGA
AAATCTACTCATGCCGAGGACAGTTCTCTATTTCCTCTATCTATACAAGGTCGTATTA
GAGGTCGAGGTTTCTTAAAAGGGTTGCTAGACGGATTAATGAGAGCAAGTTGCTGC
CAAGTAATACACCGGAGAAGTCTGGCTCATTTGAAGAGGCCGGCCAACGCAGTGTA
CGGAGGTTTGATTTACTTGATTGATAAATTGAGTGTATCACCTCCATTCCTTTCTCTT
ACTAGATCAGGACCTATTAGAGACGAATTAGAAACGATTCCCCACAAGATCCCAAC
CTCCTATCCGACAAGCAACCGTGATATGGGGGTGATTGTCAGAAATTACTTCAAATA
CCAATGCCGTCTAATTGAAAAGGGAAAATACAGATCACATTATTCACAATTATGGTT
ATTCTCAGATGTCTTATCCATAGACTTCATTGGACCATTCTCTATTTCCACCACCCTC
TTGCAAATCCTATACAAGCCATTTTTATCTGGGAAAGATAAGAATGAGTTGAGAGAG
CTGGCAAATCTTTCTTCATTGCTAAGATCAGGAGAGGGGTGGGAAGACATACATGTG
AAATTCTTCACCAAGGACATATTATTGTGTCCAGAGGAAATCAGACATGCTTGCAAG
TTCGGGATTGCTAAGGATAATAATAAAGACATGAGCTATCCCCCTTGGGGAAGGGA
ATCCAGAGGGACAATTACAACAATCCCTGTTTATTATACGACCACCCCTTACCCAAA
GATGCTAGAGATGCCTCCAAGAATCCAAAATCCCCTGCTGTCCGGAATCAGGTTGGG
CCAATTACCAACTGGCGCTCATTATAAAATTCGGAGTATATTACATGGAATGGGAAT
CCATTACAGGGACTTCTTGAGTTGTGGAGACGGCTCCGGAGGGATGACTGCTGCATT
ACTACGAGAAAATGTGCATAGCAGAGGAATATTCAATAGTCTGTTAGAATTATCAG
GGTCAGTCATGCGAGGCGCCTCTCCTGAGCCCCCCAGTGCCCTAGAAACTTTAGGAG
GAGATAAATCGAGATGTGTAAATGGTGAAACATGTTGGGAATATCCATCTGACTTAT
GTGACCCAAGGACTTGGGACTATTTCCTCCGACTCAAAGCAGGCTTGGGGCTTCAAA
TTGATTTAATTGTAATGGATATGGAAGTTCGGGATTCTTCTACTAGCCTGAAAATTG
AGACGAATGTTAGAAATTATGTGCACCGGATTTTGGATGAGCAAGGAGTTTTAATCT
ACAAGACTTATGGAACATATATTTGTGAGAGCGAAAAGAATGCAGTAACAATCCTT
GGTCCCATGTTCAAGACGGTCGACTTAGTTCAAACAGAATTTAGTAGTTCTCAAACG
TCTGAAGTATATATGGTATGTAAAGGTTTGAAGAAATTAATCGATGAACCCAATCCC
GATTGGTCTTCCATCAATGAATCCTGGAAAAACCTGTACGCATTCCAGTCATCAGAA
CAGGAATTTGCCAGAGCAAAGAAGGTTAGTACATACTTTACCTTGACAGGTATTCCC
TCCCAATTCATTCCTGATCCTTTTGTAAACATTGAGACTATGCTACAAATATTCGGAG
TACCCACGGGTGTGTCTCATGCGGCTGCCTTAAAATCATCTGATAGACCTGCAGATT
TATTGACCATTAGCCTTTTTTATATGGCGATTATATCGTATTATAACATCAATCATAT
CAGAGTAGGACCGATACCTCCGAACCCCCCATCAGATGGAATTGCACAAAATGTGG
GGATCGCTATAACTGGTATAAGCTTTTGGCTGAGTTTGATGGAGAAAGACATTCCAC
TATATCAACAGTGTTTAGCAGTTATCCAGCAATCATTCCCGATTAGGTGGGAGGCTG
TTTCAGTAAAAGGAGGATACAAGCAGAAGTGGAGTACTAGAGGTGATGGGCTCCCA
AAAGATACCCGAATTTCAGACTCCTTGGCCCCAATCGGGAACTGGATCAGATCTCTG
GAATTGGTCCGAAACCAAGTTCGTCTAAATCCATTCAATGAGATCTTGTTCAATCAG
CTATGTCGTACAGTGGATAATCATTTGAAATGGTCAAATTTGCGAAGAAACACAGG
AATGATTGAATGGATCAATAGACGAATTTCAAAAGAAGACCGGTCTATACTGATGTT
GAAGAGTGACCTACACGAGGAAAACTCTTGGAGAGATTAAAAAATCATGAGGAGAC
TCCAAACTTTAAGTATGAAAAAAACTTTGATCCTTAAGACCCTCTTGTGGTTTTTATT
TTTTATCTGGTTTTGTGGTCTTCGT.

The nucleotide sequences and vectors of the invention may be delivered to cells, for example if the aim is to express the MARV antigens in cells in order to produce and isolate the expressed proteins, such as from cells grown in culture. For expressing the antigen in cells any suitable transfection, transformation, or gene delivery methods may be used. Such methods are well known by those skilled in the art, and one of skill in the art would readily be able to select a suitable method depending on the nature of the nucleotide sequences, vectors, and cell types used. For example, transfection, transformation, microinjection, infection, electroporation, lipofection, or liposome-mediated delivery could be used. Expression of the antigen may be carried out in any suitable type of host cells, such as bacterial cells, yeast, insect cells, and mammalian cells. The antibodies of the invention may also be expressed using including in vitro transcription/translation systems. All of such methods are well known by those skilled in the art, and one of skill in the art would readily be able to select a suitable method depending on the nature of the nucleotide sequences, vectors, and cell types used.

Alternatively, methods which are well known to those skilled in the art may be used to construct expression vectors containing nucleic acid molecules that encode the polypeptide or homologs or derivatives thereof under appropriate transcriptional/translational control signals, for expression. These methods include in vitro recombinant DNA techniques, synthetic techniques and in vivo recombination/genetic recombination. See, for example, the techniques described in Maniatis et al., 1989.

The compounds or compositions may be administered orally, subcutaneously or parenterally including intravenous, intraarterial, intramuscular (IM), intraperitoneally, and intranasal administration as well as intrathecal and infusion techniques. IM is preferred, but other routes can be used such as subcutaneous or application to mucosal surfaces in the nose or mouth.

In an advantageous embodiment, the administration is IM. The dosage is measured in PFUs. The present invention illustrates that low doses of the vaccine are as effective as higher doses. Applicants have demonstrated that a single vaccination dose of 2×10⁷ PFUs down to 200 PFUs of the rVSVΔG-MARV-GP vaccine were effective in protecting cynomolgus macaques against Marburg virus infection. The dosage administration may be about 10²-10⁷ PFUs. Advantageously, the dosage may be about 10⁴, 10⁵, 10⁶ PFUs.

Combination vaccines are also contemplated. The Marburg virus vaccine of the present invention may be combined with another vaccine, such as a vaccine against a virus in the family Filoviridae. Ebola (EBOV), Sudan (SUDV) and Marburg (MARV) viruses are the three filoviruses which have caused the most fatalities in humans. It has been demonstrated that combinations of MARV or SUDV with the EBOV vaccine can be formulated yielding bivalent vaccines retaining full efficacy (Lehrer et al., Front Immunol. 2021 Aug. 18; 12:703986. doi: 10.3389/fimmu.2021.703986. eCollection 2021).

It is noted that humans may require higher vaccine dosages than mice or other experimental animals to elicit an effective immune response. The doses may be single doses or multiple doses over a period of time, but single doses are preferred. Thus, one may scale up from animal experiments, e.g., rats, mice, and the like, to humans, by techniques from this disclosure and documents cited herein and the knowledge in the art, without undue experimentation.

In another embodiment, the immunization of primates is also considered. For the immunization of nonhuman primates, oral administration (such as via bait drop) is contemplated. Non-human primates that may be immunized by the vaccine of the present invention include, but are not limited to, chimpanzees and bonobos, gorillas, orangutans, gibbons and monkeys. The immunization against other animals carrying the Marburg virus (such as bats) is also contemplated. In this instance, a bait drop is contemplated. In one embodiment, the bait drop may comprise a hollow plastic packet. In another embodiment, the composition may be inserted in the hollow polymer cube. For example, the bait drop can comprise a fishmeal polymer cube (1.25 inches by 0.75 inches) that is hollow. A sachet, or plastic packet, containing the vaccine can be inserted into the hollow area of the bait and sealed with wax.

When administering a therapeutic of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier may be a solvent or dispersing medium containing, for example, water, ethanol, polyol (for example, glycerol, propylene glycol, liquid polyethylene glycol, and the like), suitable mixtures thereof, and vegetable oils.

Additionally, various additives which enhance the stability, sterility, and isotonicity of the compositions, including antimicrobial preservatives, antioxidants, chelating agents, and buffers, may be added. Prevention of the action of microorganisms may be ensured by various antibacterial and antifungal agents, for example, parabens, chlorobutanol, sorbic acid, and the like. In many cases, it will be desirable to include isotonic agents, for example, sugars, sodium chloride, and the like. Prolonged absorption of the injectable pharmaceutical form may be brought about by the use of agents delaying absorption, for example, aluminum monostearate and gelatin. According to the present invention, however, any vehicle, diluent, or additive used would have to be compatible with the vaccine preparation. Formulations that stabilize the virus are also contemplated. Additions such as, but not limited to, carbohydrates (such as sucrose or trehalose), gelatin, hydrolyzed gelatin, amino acids, etc. are contemplated.

Sterile injectable solutions may be prepared by incorporating the compounds utilized in practicing the present invention in the required amount of the appropriate buffered solution with various amounts of the other ingredients, as desired.

A pharmacological formulation of the present invention, e.g., which may comprise a therapeutic compound or polypeptide of the present invention, may be administered to the patient in an injectable formulation containing any compatible carrier, such as various vehicles, adjuvants, additives, and diluents; or the compounds utilized in the present invention may be administered parenterally to the patient in the form of or polymer matrices, liposomes, and microspheres.

A pharmacological formulation of the compound and composition which may comprise a polypeptide utilized in the present invention may be administered orally to the patient. Conventional methods such as administering the compounds in tablets, suspensions, solutions, emulsions, capsules, powders, syrups and the like are usable. Known techniques, which deliver the compound orally or intravenously and retain the biological activity, are preferred.

In one embodiment, a formulation of the present invention may be administered initially, and thereafter maintained by further administration. For instance, a formulation of the invention may be administered in one type of composition and thereafter further administered in a different or the same type of composition. For example, a formulation of the invention may be administered by intravenous injection to bring blood levels to a suitable level. The patient's levels are then maintained by an oral dosage form, although other forms of administration, dependent upon the patient's condition, may be used. In the instance of a vaccine composition, the vaccine may be administered as a single dose, or the vaccine may incorporate set booster doses.

The quantity to be administered will vary for the patient being treated and whether the administration is for treatment or prevention and will vary from about 10²-10⁷ plaque-forming units (PFUs) of the vaccine. The administering may be about 10⁴ PFU, about 10⁵ PFU or about 10⁶ PFU of the vaccine.

Of course, for any composition to be administered to an animal or human, including the components thereof, and for any particular method of administration, it is preferred to determine therefore: toxicity, such as by determining the lethal dose (LD) and LD₅₀ in a suitable animal model e.g., rodent such as mouse; and, the dosage of the composition(s), concentration of components therein and timing of administering the composition(s), which elicit a suitable immunological response, such as by titrations of sera and analysis thereof for antibodies or antigens, e.g., by ELISA and/or Rapid Fluorescent Foci Inhibition Test (RFFIT) analysis. Such determinations do not require undue experimentation from the knowledge of the skilled artisan, this disclosure and the documents cited herein. And, the time for sequential administrations may be ascertained without undue experimentation. For instance, dosages may be readily ascertained by those skilled in the art from this disclosure and the knowledge in the art. Thus, the skilled artisan may readily determine the amount of compound and optional additives, vehicles, and/or carrier in compositions and to be administered in methods of the invention. Typically, an adjuvant or additive is commonly used as 0.001 to 50 wt % solution in phosphate buffered saline, and the active ingredient is present in the order of micrograms to milligrams, such as about 0.0001 to about 5 wt %, preferably about 0.0001 to about 1 wt %, most preferably about 0.0001 to about 0.05 wt % or about 0.001 to about 20 wt %, preferably about 0.01 to about 10 wt %, and most preferably about 0.05 to about 5 wt %. Such determinations do not require undue experimentation from the knowledge of the skilled artisan, this disclosure and the documents cited herein. And, the time for sequential administrations may be ascertained without undue experimentation.

Examples of compositions which may comprise a therapeutic of the invention include liquid preparations for orifice, e.g., oral, nasal, anal, vaginal, peroral, intragastric, mucosal (e.g., perlingual, alveolar, gingival, olfactory or respiratory mucosa) etc., administration such as suspensions, syrups or elixirs; and, preparations for parenteral, subcutaneous, intradermal, intramuscular or intravenous administration (e.g., injectable administration), such as sterile suspensions or emulsions. Such compositions may be in admixture with a suitable carrier, diluent, or excipient such as sterile water, physiological saline, glucose or the like. The compositions may also be lyophilized. The compositions may contain auxiliary substances such as wetting or emulsifying agents, pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Compositions of the invention, are conveniently provided as liquid preparations, e.g., isotonic aqueous solutions, suspensions, emulsions or viscous compositions which may be buffered to a selected pH. If digestive tract absorption is preferred, compositions of the invention may be in the “solid” form of pills, tablets, capsules, caplets and the like, including “solid” preparations which are time-released or which have a liquid filling, e.g., gelatin covered liquid, whereby the gelatin is dissolved in the stomach for delivery to the gut. If nasal or respiratory (mucosal) administration is desired, compositions may be in a form and dispensed by a squeeze spray dispenser, pump dispenser or aerosol dispenser. Aerosols are usually under pressure by means of a hydrocarbon. Pump dispensers may preferably dispense a metered dose or, a dose having a particular particle size.

Compositions of the invention may contain pharmaceutically acceptable flavors and/or colors for rendering them more appealing, especially if they are administered orally. The viscous compositions may be in the form of gels, lotions, ointments, creams and the like (e.g., for transdermal administration) and will typically contain a sufficient amount of a thickening agent so that the viscosity is from about 2,500 to 6,500 cps, although more viscous compositions, even up to 10,000 cps may be employed. Viscous compositions have a viscosity preferably of 2,500 to 5,000 cps, since above that range they become more difficult to administer. However, above that range, the compositions may approach solid or gelatin forms, which are then easily administered as a swallowed pill for oral ingestion.

Liquid preparations are normally easier to prepare than gels, other viscous compositions, and solid compositions. Additionally, liquid compositions are somewhat more convenient to administer, especially by injection or orally. Viscous compositions, on the other hand, may be formulated within the appropriate viscosity range to provide longer contact periods with mucosa, such as the lining of the stomach or nasal mucosa.

Obviously, the choice of suitable carriers and other additives will depend on the exact route of administration and the nature of the particular dosage form, e.g., liquid dosage form (e.g., whether the composition is to be formulated into a solution, a suspension, gel or another liquid form), or solid dosage form (e.g., whether the composition is to be formulated into a pill, tablet, capsule, caplet, time release form or liquid-filled form).

Solutions, suspensions and gels, normally contain a major amount of water (preferably purified water) in addition to the active compound. Minor amounts of other ingredients such as pH adjusters (e.g., a base such as NaOH), emulsifiers or dispersing agents, buffering agents, preservatives, wetting agents, jelling agents, (e.g., methylcellulose), colors and/or flavors may also be present. The compositions may be isotonic, i.e., it may have the same osmotic pressure as blood and lacrimal fluid.

The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions may be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount that will achieve the selected viscosity. Viscous compositions are normally prepared from solutions by the addition of such thickening agents.

A pharmaceutically acceptable preservative may be employed to increase the shelf-life of the compositions. Benzyl alcohol may be suitable, although a variety of preservatives including, for example, parabens, thimerosal, chlorobutanol, or benzalkonium chloride may also be employed. A suitable concentration of the preservative will be from 0.02% to 2% based on the total weight although there may be appreciable variation depending upon the agent selected.

Those skilled in the art will recognize that the components of the compositions should be selected to be chemically inert with respect to the active compound. This will present no problem to those skilled in chemical and pharmaceutical principles, or problems may be readily avoided by reference to standard texts or by simple experiments (not involving undue experimentation), from this disclosure and the documents cited herein.

It is generally envisaged that compounds and compositions of the invention will be administered by injection, as such compounds are to elicit anti-MARV antibodies, and the skilled artisan may, from this disclosure and the knowledge in the art, formulate compounds and compositions identified by herein methods for administration by injection and administer such compounds and compositions by injection.

The inventive compositions of this invention are prepared by mixing the ingredients following generally accepted procedures. For example the selected components may be simply mixed in a blender, or other standard device to produce a concentrated mixture which may then be adjusted to the final concentration and viscosity by the addition of water or thickening agent and possibly a buffer to control pH or an additional solute to control tonicity. Generally the pH may be from about 3 to 8.5. Compositions may be administered in dosages and by techniques well known to those skilled in the medical arts taking into consideration such factors as the age, sex, weight, and condition of the particular patient, and the composition form used for administration (e.g., solid vs. liquid). Dosages for humans or other mammals may be determined without undue experimentation by the skilled artisan, from this disclosure, the documents cited herein, and the knowledge in the art.

Suitable regimes for initial administration and further doses or for sequential administrations also are variable, may include an initial administration followed by subsequent administrations; but nonetheless, may be ascertained by the skilled artisan, from this disclosure, the documents cited herein, and the knowledge in the art.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### Example: Vaccination Against Marburg Virus Disease Using a Recombinant Vesicular Stomatitis Virus and Potential for Dose

Vaccines are needed to disrupt or prevent continued outbreaks of filoviruses in humans across Western and Central Africa, including outbreaks of MARV. As part of a filovirus vaccine product development plan, it is important to investigate dose response early in preclinical development to identify the dose range that may be optimal for safety, immunogenicity and efficacy, and perhaps demonstrate that using lower doses is feasible, which will improve product access. To determine the efficacious dose range for a manufacturing-ready live VSV vaccine vector encoding the MARV glycoprotein (rVSVΔG-MARV-GP), a dose-range study was conducted in cynomolgus macaques, and the results showed that a single vaccination with as little as 200 PFUs was 100% efficacious against lethality and prevented development of viremia when the animals were challenged with the MARV Angola variant. rVSVΔG-MARV-GP vaccination induced MARV GP-specific serum IgG, and virus-neutralizing activity in serum was detectable in animals vaccinated with the highest doses. The data implies use of lower doses of MARV vaccine should be further investigated.

Filoviruses are a major threat to global health and continue to impact the health security and geopolitical stability of central and western Africa. A major EBOV outbreak occurred in West Africa in 2014-2016 and has been followed by a concerning frequency of outbreaks in the Democratic Republic of the Congo and Guinea [1]. Outbreaks have been caused by EBOV recrudescence-related events in non-endemic parts of West Africa as well as recent zoonotic transmission in endemic regions [2, 3]. Other filoviruses remain endemic in animal reservoirs across Africa, including MARV, SUDV, and others that cause lethal hemorrhagic fevers in humans and have similar epidemic potential to EBOV [4]. Highlighting the risk of zoonotic transmission, modeling indicates that the geographic regions that might support transmission of MARV are quite extensive [5]. Moreover, a transmission event was detected for the first time in West Africa in a patient from Guinea with no travel history [6] and most recently in Ghana [7]. Outbreaks of filoviruses including MARV will continue to happen at an accelerated rate in the future with factors such as climate change, increased inter-continental travel, population growth, and zoonotic reservoir range expansion contributing to the likelihood of future disease transmission events [8].

Vaccination against filoviruses in response to outbreaks and as a regular public health measure has the potential to help further control the health security threat to Africa. The success of the ZEBOV vaccine produced by Merck Vaccines (rVSVΔG-ZEBOV-GP marketed as ERVEBO®) has provided a strong rationale for efforts to develop other recombinant, live-attenuated vaccines based on the VSV vaccine vector technology [9, 10]. The performance of ERVEBO in outbreak environments has clearly shown that the VSV-based technology has multiple features needed for development of other effective filovirus vaccines including, 1) acceptable safety and tolerability; 2) efficacy after a single dose; and 3) rapid development of protective immunity [9-13].

In addition to the key rVSVΔG-ZEBOV-GP performance features mentioned above, it is also important to consider factors that affect access to filoviruses vaccines for populations where the viruses are endemic in Western and Central Africa. Availability of vaccine material that is safe for use in humans and can be deployed rapidly is important, as outbreaks of EBOV and other filoviruses such as MARV or SUDV cannot be forecasted with any certainty [10]. Filovirus vaccines must also be cost-efficient, and thus dose-sparing conditions and efficacy following a single dose are important to evaluate. Finally, because of the sporadic nature of filovirus outbreaks, traditional human efficacy trials are not feasible. Thus, access to new filovirus vaccines will likely require use of existing alternative regulatory pathways such as the animal rule and accelerated approval as well as the generation of innovative data packages to demonstrate adequate safety, immunogenicity, and efficacy through preclinical animal studies and human clinical trials [14]. In the case of VSV-based filovirus vaccines, this can be facilitated by the preclinical and clinical track record of rVSVΔG-ZEBOV-GP [9-11] and the extensive preclinical research conducted on MARV and other filovirus vaccines based on the rVSVΔG-ZEBOV-GP design previously [15-17].

In response to the first identified human MARV case in West Africa, the WHO convened filovirus expert groups comprised of infectious disease scientists, epidemiologists, public health experts, and vaccine developers, to put together a research and development blueprint to enhance the WHO's “Strategic Agenda for Filoviruses Research and Monitoring” (AFIRM) [18]. Central to this blueprint is understanding what experimental MARV vaccines are available and their state of development, and what preclinical data is available that supports their use in an outbreak situation. Furthermore, the blueprint will cover development of clinical trial approaches that can be utilized during a public health emergency due to a MARV outbreak. It is likely that ring vaccination strategies could be implemented as early as possible in response to an emergence of MARV or other filovirus threats, and that the availability of clinical trial material, as was the case for Ebola Zaire, would aid in the response to future filovirus outbreaks [19]. Without the availability of vaccine prepared according to GMP and ready for immediate use, the response to the 2014-2016 West African Ebola Zaire outbreak would have been much slower and had even more far-reaching consequences in terms of the toll on human lives and economically.

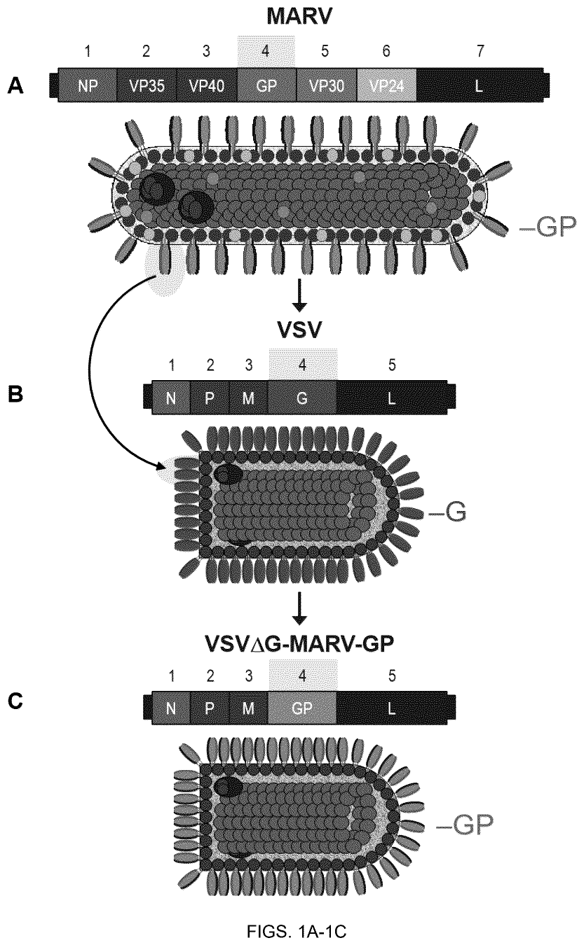

A MARV vaccine candidate (rVSVΔG-MARV-GP; FIG. 1) based on the VSV technology used for ERVEBO is currently being developed and a research vaccine has been shown to be safe and efficacious in multiple preclinical studies. To advance rVSVΔG-MARV-GP as a globally-accessible vaccine candidate for human use, Applicants regenerated a recombinant vaccine strain using conditions that would support future human vaccine development and tested it across a range of doses for immunogenicity and efficacy against MARV challenge in the cynomolgus macaque animal model for MARV disease [20]. The rVSVΔG-MARV-GP vaccine was 100% efficacious against Marburg disease caused by the Angola isolate and protected against development of MARV viremia after a single IM injection even when doses as low as 200 PFUs were used. rVSVΔG-MARV-GP vaccination induced MARV GP-specific humoral responses that can be further interrogated to better understand correlates of protection and this data will help provide an important bridge to future human safety and immunogenicity studies. The value of the VSVΔG-based MARV vaccine approach and next steps for filovirus vaccine development are discussed.

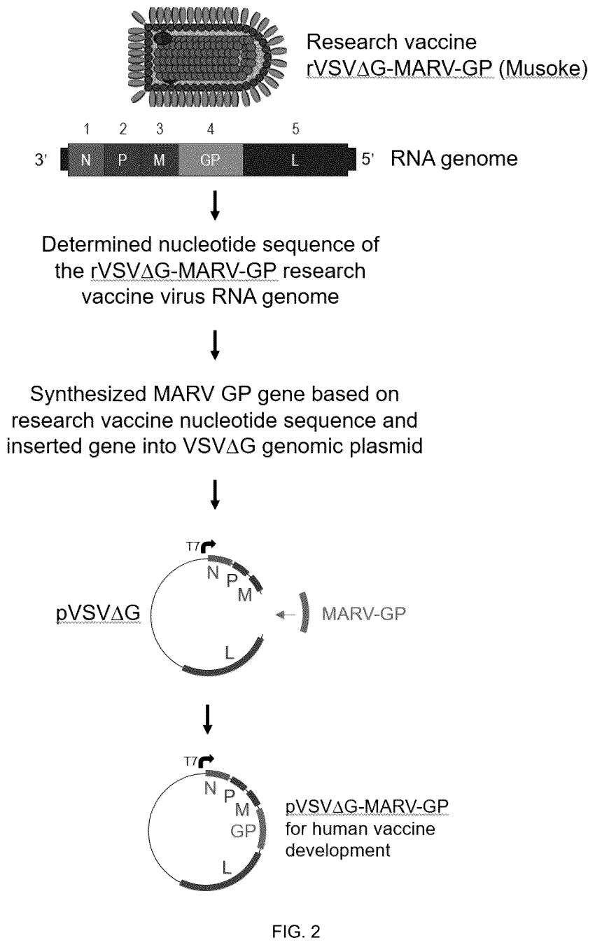

Cell culture and recombinant VSV. As summarized in FIG. 2., A research stock of rVSVΔG-MARV-GP encoding GP from the MARV Musoke strain [21], which was used in multiple previous preclinical studies [22-24], was obtained from the Public Health Agency of Canada (PHAC). The nucleotide sequence of the viral RNA genome was determined by Sanger sequencing as described before [25] after which a DNA fragment encoding the MARV-GP gene was synthesized at GenScript (Piscataway). The GP gene was then transferred into the VSV Indiana genomic plasmid as described earlier [21]. The nucleotide sequence of the new genomic clone was confirmed by Sanger sequencing. The genomic plasmid DNA was propagated and purified using animal product-free medium and reagents.

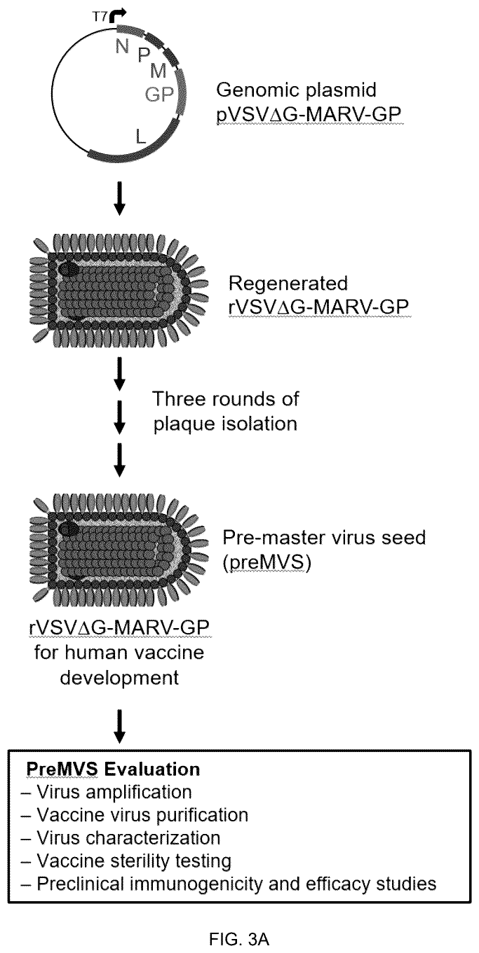

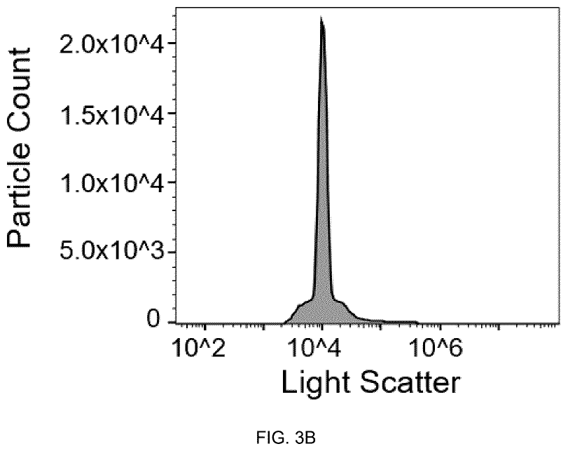

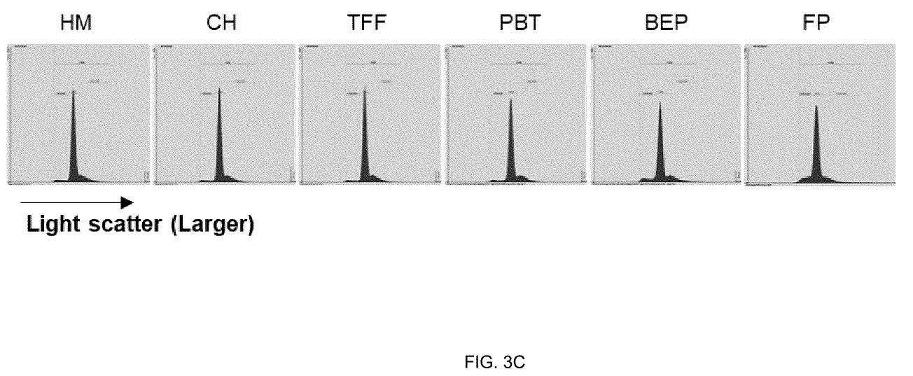

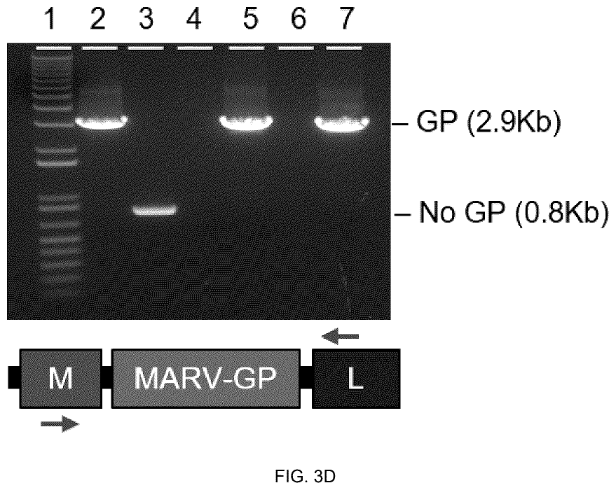

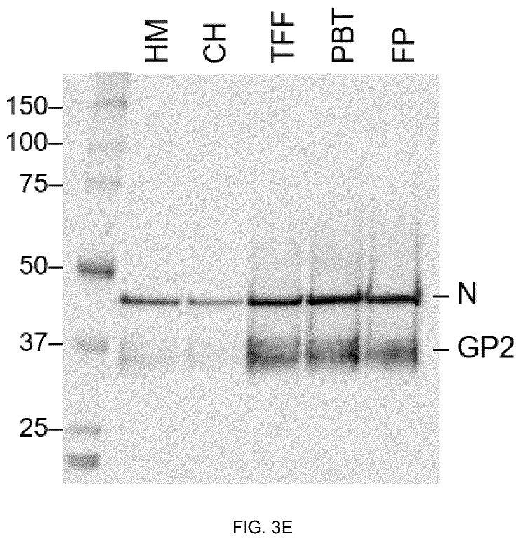

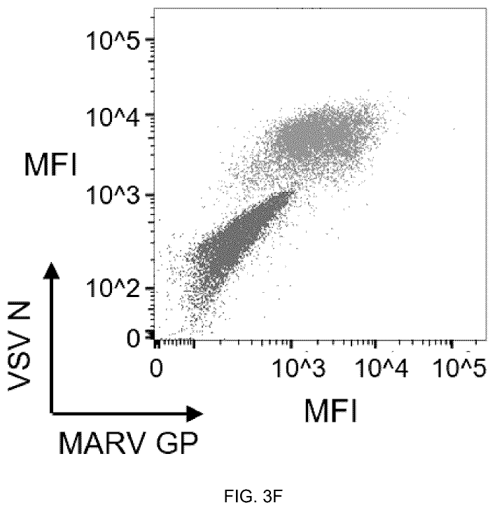

Recovery of rVSVΔG-MARV-GP from plasmid DNA was initiated by electroporating Vero cells [25]. A cell bank used before that was qualified for human vaccine production [26] was used at all stages of recombinant virus work (FIG. 3). This cell bank was derived from the WHO working cell bank (WHO 10-87) deposited at the European Collection of Authenticated Cell Cultures (Vero [WHO], ECACC 88020401). Vero cells were cultured in Dulbecco's modified Eagle medium (DMEM; Sigma) supplemented with 4 mM L-glutamine and 10% gamma-irradiated fetal bovine serum (FBS; Sigma Aldrich). Electroporation was conducted using methods modified from those described before [25, 27, 28]. In brief, ˜2.5×10⁷ cells were electroporated (low voltage mode, 3 pulses, 70 milliseconds, 140V at 900 millisecond intervals) using a BTX830 apparatus (Harvard Apparatus) and then were cultured at 37° C. in 5% CO₂ and 85% humidity. Cell supernatant was harvested 3 days post-electroporation and used to infect Vero cell monolayers cultured in supplemented DMEM to amplify the new recombinant virus. Two days later, medium containing virus was collected, and stored at <60° C. After confirming the rescued virus population had the expected genomic consensus sequence, three rounds of plaque isolation were performed.

Isolated virus plaques were picked from infected Vero cell monolayers overlaid with DMEM containing the supplements mentioned above with 2% FBS and 0.5% agarose (Lonza). Virus from multiple individual plaques were amplified in Vero cells, after which genomic sequence analysis were performed to identify lead candidates with the expected genome sequence. Lead candidates were then subjected to two additional rounds of plaque isolation after which selected candidates were used to infect Vero cells cultured in 5-layer Cell Stacks (Corning). Approximately 40 hours after infection medium was harvested and clarified by low-speed centrifugation before being stored in aliquots at <−60° C. Virus stocks were characterized using multiple assays to confirm expected genomic sequence, MARV GP expression, and no contaminants presence. The selected candidate was then designated as the preMVS.

Vaccine material for preclinical studies derived from the preMVS was produced in Vero cell cultures and purified with a process based on TFF. Briefly, Vero cells were seeded in a 5-layer Cell Stack with DMEM supplemented as described above and incubated for 72 hours to achieve a monolayer that was near confluent. Before infection, the cell monolayer was washed two times with DMEM before adding 375 ml of Virus-Production Serum-Free Medium (VP-SFM; Thermo Fisher Scientific) containing virus to achieve a multiplicity of infection (MOI) of 0.001. At 40 hours after infection, medium containing virus was harvested and subsequently clarified by sequential filtration with a 1.2 μm filter (Sartorius) followed by a 0.8/0.45 μm depth filter (Pall Corporation). TFF was used to concentrate and further purify the virus using a 750 kDa hollow fiber membrane (Repligen Corporation). This was followed by addition of MgCl₂ (InVitrogen, Thermo Fisher Scientific) to a final concentration of 1.5 mM and benzonase (200 U/ml; Sigma-Aldrich) while continuing TFF for 30 minutes at room temperature. Buffer exchange was performed with 50 mM Tris-HCl, 150 mM NaCl, and 10% sucrose buffer, pH 8.0. Purified virus vaccine candidate was aliquoted and stored at <−80° C. FIG. 3 illustrates some of the characterization performed with the vaccine candidate, such as flow virometry, genome integrity, and MARV GP expression.

Flow-virometry (Apogee). VSVΔG-MARV-GP vaccine purified virus was run on A60-MicroPLUS Apogee flow cytometer using highly purified Milli-Q water as sheath fluid. The sample was diluted 1:300 in sterile HBSS buffer and ran at 1.5 μL/minute with autocycler set to 200,000 total events. 405 nm violet laser was set to 150 mW and Large-Angle Light Scatter detector was used to successfully resolve VSV virus peak profile.

Genome integrity analysis and sequencing. Genome integrity of MARV GP was assessed by RT-PCR with Superscript IV One-Step RT-PCR System (Invitrogen), where forward and reverse primers were in the VSV M and L genes, respectively. RT-PCR was performed at 60° C. for 10 min and 98° C. for 2 mins. Followed by 40 cycles of 98° C. for 10s, 70° C. for 10s, and 72° C. for 1.5 mins with final extension at 72° C. for 5 mins. A 2.9 Kb band was detected in an 0.8% agarose gel and excised for DNA extraction (Qiagen). Sanger sequencing was performed with BigDye Terminator v3.1 Cycle Sequencing Kit (ThermoFisher Scientific) and BigDye XTerminator™ Purification Kit (ThermoFisher Scientific) with ABI 3500XL Genetic Analyzer (ThermoFisher Scientific).

Analysis of GP expression. Incorporation of GP in virions was monitored at multiple stages of production using Western blotting and methods similar to those described earlier [25]. Samples containing rVSVΔG-MARV-GP were denatured and separated using 4-12% Bis-Tris denaturing polyacrylamide gels (Invitrogen) and transferred with iBLOT2 system to nitrocellulose membranes (Invitrogen). To detect virion proteins, rabbit polyclonal anti-MARV GP (Cat. 0303-007_IBT Bioservices) and anti-VSV N (produced in house [29]) were used as primary antibodies, and goat anti-rabbit HRP (Santa Cruz) as secondary antibody. Signals were detected using SuperSignal West Femto Maximum Sensitivity (ThermoFisher Scientific) and the ChemiDoc Imaging System (BioRad).

Flow cytometry was used to assess cell-surface expression of MARV GP and intracellular expression of VSV N. Adherent infected cells were detached from plates 48hpi by scraping them into a wash solution containing PBS supplemented with 0.5% BSA (PBS/BSA). Cell suspension was distributed into a 96 deep-well tissue culture plate before collection by low-speed centrifugation for 5 mins at 860 xg. For MARV GP staining on the cell surface, cells were initially resuspended in PBS/BSA containing mouse monoclonal anti-MARV GP (Cat. 0203-023, 5C1, IBT Bioservices), rabbit polyclonal anti-MARV GP (Cat. 0303-007, IBT Bioservices) orpan-filovirus-chimeric anti-GP mAb (Cat. 0200-003, IBT Bioservices) at a final concentration of 1 μg/ml and incubated at room temperature for 25 minutes. Cells were collected by centrifugation, resuspended in PBS/BSA, and centrifugation was repeated to remove free anti-GP antibodies. Pelleted cells were resuspended in Cytofix/Cytoperm Solution (BD Biosciences) and incubated for 20 mins at 4° C. in the dark. Permeabilized cells were collected by centrifugation and resuspended in Perm/Wash Buffer (BD Biosciences) before repeating centrifugation. To stain intracellular VSV N, cells were resuspended in Perm/Wash Buffer containing anti-VSV N mouse monoclonal antibody (Cat. EB0009,10G4, Kerafast) at 1 μg/ml final concentration, and incubated in the dark at room temperature for 25 mins. Following incubation, the cells were collected and washed with Perm/Wash Solution as described above, cells were resuspended in a goat anti-mouse IgG1 or goat anti-Rabbit Alexa 555 and goat anti-mouse IgG2a Alexa 647 secondary antibody solution (ThermoFisher Cats. A-21127, A-32732, A21241 respectively, ThermoFisher) and incubated in the dark at room temperature for 25 minutes. Perm/Wash Buffer (BD Biosciences) was used for one wash step and resupension of the cells. Flow cytometry was performed with BD SORP LSRII flow cytometer (BD Biosciences).

VSVΔG-MARV-GP vaccination. Vaccination was performed in ABSL2 suites at the University of Texas Medical Branch (UTMB). The study design was approved by the UTMB Institutional Biosafety Committee (IACUC), and all animal research was conducted in compliance with the UTMB IACUC, Animal Welfare Act, and other federal statutes and regulations relating to animal care. The UTMB animal research facility is fully accredited by the Association for Assessment and Accreditation of Laboratory Animal Care.

This study contained 6 groups (n=4 per group). Five groups were vaccinated with different doses of rVSV-MARV-GP ranging from 2×10⁷ PFUs down to 200 PFUs. The control group was vaccinated with a vaccine prepared from a preMVS developed from a similar VSV-based vaccine (IAVI unpublished) that expressed the Lassa virus glycoprotein (rVSVΔG-LASV-GPC [21, 30]). Animals received one IM injection in the quadriceps. Diluted unused vaccine material was back tittered to confirm delivery of the targeted vaccine dose.

Analysis of anti-MARV GP serum IgG. Blood was drawn via peripheral venipuncture using serum separator tubes (Greiner Bio-One, Monroe, NC) prior to, and on days 10 and 27 post vaccination. Serum was stored frozen (−20° C.) until analysis by indirect ELISA or plaque reduction assay to assess neutralizing antibody titers. Anti-MARV GP IgG endpoint titers were quantified using ELISA plates (96 half-well plates; Corning) coated overnight at 4° C. with a soluble form of recombinant MARV Angola GP (obtained from the US Department of Defense, Joint Program Executive Office for Chemical, Biological, Radiological and Nuclear Defense, CBRN-JPEO) diluted to 1 μg per ml in ELISA Coating Buffer (Biolegend, San Diego, CA). After coating, the plates were blocked with blocking buffer (3% BSA in PBS with 0.05% Tween-20) for 1.5 hours at 37° C. and then washed with 150 μl of wash buffer (PBS containing 0.05% Tween-20). Serum samples were then added in three-fold dilutions starting at a 1:100 dilution and incubated for 1 hour at 37° C. Following incubation, the plates were washed and incubated with anti-human IgG (H+L)-HRP (Jackson Immunoresearch) at 1:6,000 dilution for 1 hour at 37° C., washed again, developed using 1-Step Ultra TMB substrate (Thermo Fisher Scientific, Waltham MA), and stopped with 5N Sulfuric acid (Thermo Fisher Scientific, Waltham MA) after 10 minutes. Plates were read within 30 minutes at 450 nm with a Molecular Devices (San Jose, CA) VersaMax Microplate Reader using SoftMax Pro GxP Data Acquisition Software. Serum from unvaccinated animals or serum taken prior to vaccination was included to determine assay background. Titers were defined as the serum dilution resulting in an absorbance >2-times the standard deviation of background wells. A commercially available anti-MARV GP antibody (IBT Bioservices, Rockville MD) served as a positive control in the assay.

Virus-neutralizing anti-MARV GP serum antibodies (nAbs) were quantified using rVSVΔG-MARV-GP (Musoke) as the target virus. Serum collected 27 days after vaccination was heat inactivated at 56° C. for 30 minutes, clarified by centrifugation at 9,300×g for 10 minutes, then diluted serially from 1:20 to 1:327,680 and incubated 1 hour on a shake platform at 300 RPM at 37° C. incubator with appropriate amounts of rVSVΔG-MARV-GP to produce about 100 plaques per well. After incubation, the serum-virus mixture was used to infect Vero cell monolayers in 96-well tissue culture plates. Following a 2-hour incubation on a shake platform at 300 RPM in a 37° C. incubator, the cells were overlaid with DMEM (ThermoFisher Scientific) containing 1% FBS and 0.5% methylcellulose (ThermoFisher Scientific). Plaques were allowed to develop for 44 hours at 37° C. before the methylcellulose overlay was removed and cells were fixed with 7% v/v formaldehyde prepared in water (200 μl of per well) and incubating for 1 hour at room temperature. Plaques were stained by adding 200 μl of crystal violet solution (0.33% in water) per well and incubating for 1 hour at room temperature. Staining solution was removed, and the plaques were rinsed and dried before plaques were counted using a Cytation 5 Imager Gen5 3.08 software (Agilent). The lowest serum dilution that decreased plaques by 50% or more was reported.

MARV challenge virus and vaccine efficacy. Challenge virus was prepared at UTMB using MARV Angola (200501379) isolated from an 8-month-old female patient in Uige, Angola. A challenge virus stock was developed from virus obtained from the CDC (CDC 810820) that was passaged twice in Vero E6 cells at UTMB [31]. On day 28 post-vaccination macaques were infected with 10³ PFUs by IM injection in the quadriceps. Animals were monitored daily and scored for MARV disease progression using a humane endpoint filovirus disease scoring sheet approved by the UTMB IACUC. The scoring changes measured from baseline included posture and activity level, attitude and behavior, food intake, respiration, and disease manifestations, such as visible rash, hemorrhage, ecchymosis, or flushed skin. Animals were also monitored for central nervous system abnormalities. A score of ≥10 indicated that an animal met the criteria for euthanasia. Blood was collected on days 4, 7, 10, 11, 13, 15, 21 and 28 after MARV challenge for evaluation of blood chemistries and quantification of infectious MARV. The UTMB facilities are accredited by the Association for Assessment and Accreditation of Laboratory Animal Care International and adhere to principles specified in the eighth edition of the Guide for the Care and Use of Laboratory Animals, National Research Council.

Infectious MARV in blood (viremia) was quantified using plasma collected from macaques and plaque assay [30]. Briefly, increasing ten-fold dilutions of plasma samples were allowed to infect Vero E6 monolayers (ATCC, Manassas, VA) in duplicate wells (200 l per well). The limit of detection from serum was 25 PFU/ml.

To monitor MARV genomes in blood (RNAemia) by RT-qPCR, RNA was isolated from whole blood with the viral RNA mini-kit (Qiagen) using 100 l of blood mixed with 600 l of viral lysis buffer AVL [32]. Primers targeting the NP gene of MARV were used for real-time quantitative PCR (RT-qPCR) with a 6-carboxyfluorescein (6FAM)-5′-CCCATAAGGTCACCCTCTT-3′ (SEQ ID NO: 3)-6 carboxy-tetramethylrhodamine (TAMRA) probe. Thermocycler run settings were 50° C. for 10 min; 95° C. for 10 s; and 40 cycles of 95° C. for 10 s plus 59° C. for 30 s. Primers were synthesized by Integrated DNA Technologies and labeled probes were prepared by Life Technologies. MARV genomes in samples were calculated using a genome equivalent standard. The limit of detection for this assay is 1,000 copies/ml.

Generation of a rVSVΔG-MARV-GP to support human vaccine development. The rVSVΔG-MARV-GP [21, 23] vaccine is based on a replication-competent chimeric virus design (FIG. 1) in which the gene encoding the natural VSV glycoprotein (G) is deleted (VSVΔG) and replaced with coding sequence for a functional glycoprotein from a heterologous virus [21, 33]. The rVSVΔG-MARV-GP genomic clone was generated using the genomic plasmid for a lab-adapted VSV serotype Indiana [34, 35] and GP coding sequence from the MARV Musoke strain ([21, 23]). To generate a rVSVΔG-MARV-GP strain suitable for human vaccine development, a new recombinant virus was regenerated from plasmid DNA as described in the Methods (FIG. 2).

Applicants chose to advance VSVΔG-MARV-GP expressing the MARV Musoke GP as a candidate human vaccine for multiple important reasons, including: 1) a single vaccination with the VSVΔG-MARV-GP Musoke vaccine was shown to be highly efficacious and it rapidly induced protective immunity in macaques [23, 36, 37]; 2) the protective immunity was durable [38]; 3) preclinical studies indicated that vaccination induced immunity that could protect macaques against MARV Musoke, MARV Angola, or Ravn virus [22, 23]; 4) preclinical studies indicated that the vaccine could induce protective immunity active against aerosolized MARV [39]; and 5) the rVSVΔG-MARV-GP Musoke vaccine along with a research rVSVΔG-ZEBOV-GP vaccine were evaluated in a neurovirulence study conducted in macaques, which demonstrated lack of neurovirulence potential providing valuable safety data for advancing a vaccine for human use [24].

After a new rVSVΔG-MARV-GP strain was recovered from plasmid DNA, multiple clonal virus isolates were generated by conducting three rounds of plaque isolation (Methods and FIG. 3A) while maintaining laboratory and documentation practices necessary to support human vaccine development. Several lead candidates subsequently were selected based on comparing the research virus [21] genomic nucleotide sequence Applicants determined to the clonal virus isolates, and confirmation of GP expression by Western blotting and flow cytometry (data not shown). Following amplification and banking of multiple seed stocks designated as candidate preMVSs, the preMVS candidates were analyzed using multiple assays described below to select the lead candidate.

The assays and approach used to evaluate the preMVS candidates is summarized in FIG. 3. Virus from the preMVS candidates was subjected to serial propagation in Vero cells to mimic manufacturing amplification during which titers were monitored by plaque assay (data not shown) and genetic stability was assessed using genomic nucleotide sequencing and an RT-PCR assay like shown in FIG. 3D, which detected the intact GP gene but also confirmed that no minor populations of unexpected GP deletion variants were present. GP expression also was confirmed by Western blot using whole-cell lysates (FIG. 3E) and by assessing expression on the cell surface using flow cytometry (FIG. 3F).

Virus derived from several preMVS candidates also was amplified and purified to produce preclinical vaccine material and evaluate how each preMVS performed during the production process. Vaccine material was prepared by infecting Vero cells after which virus was purified and concentrated using a scalable method based on TFF (see Methods) that was aligned with the expected manufacturing process. In addition to the assays described in FIG. 3A, D-F, Applicants also used nanoflow cytometry to quantify virion particles (FIG. 3B and C) during all stages of preclinical vaccine production process to ensure that a single predominant peak of virions was detected and that smaller particles or larger aggregates did not accumulate.

Based on the performance of the preMVS candidates in the laboratory assessment briefly summarized above, a lead preMVS was selected and was transferred to Applicants' GMP manufacturing partner. A qualified master virus seed (MVS) has since been produced to support manufacturing. The preclinical vaccine material produced from the lead preMVS also was used for the preclinical vaccine efficacy study described below.

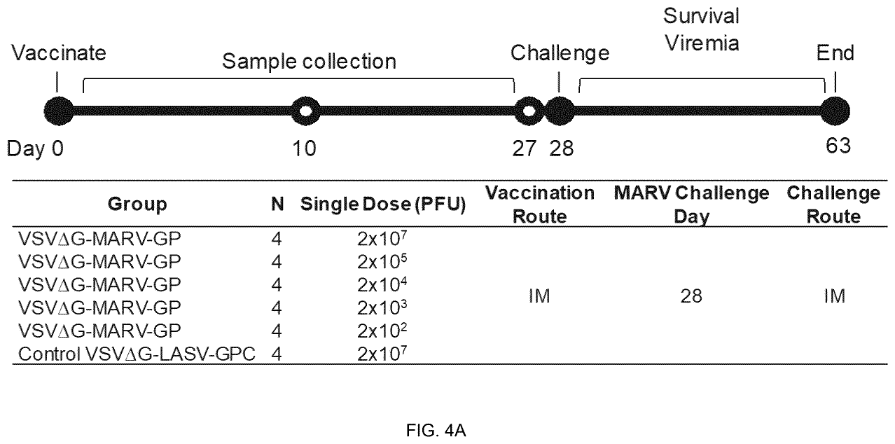

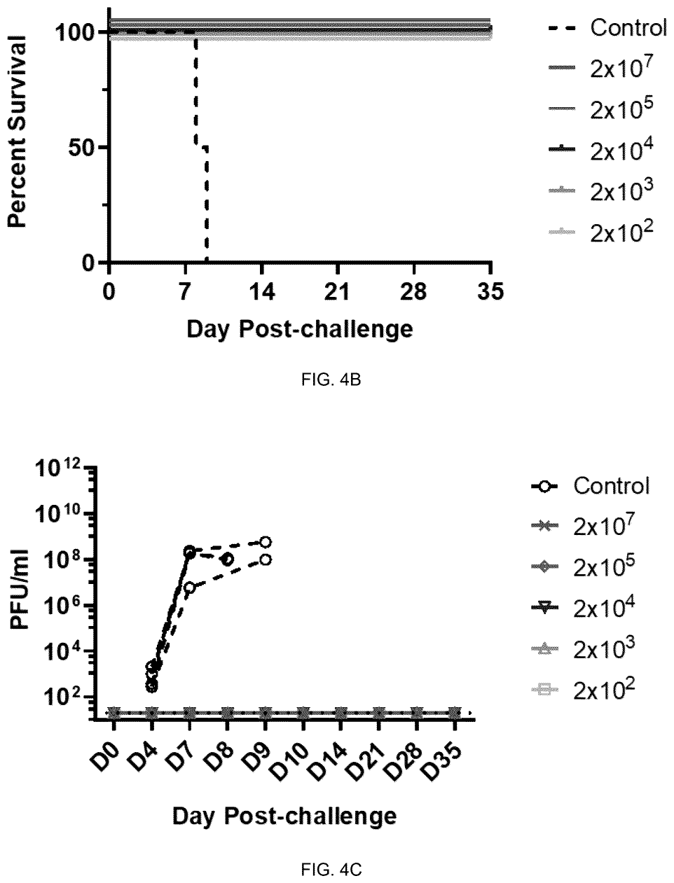

A single vaccination with a wide range of rVSVΔG-MARV-GP(Musoke) doses protects from MARV Anogla challenge. A preclinical research study (FIG. 4A) was conducted in cynomolgus macaques to investigate the dose range over which rVSVΔG-MARV-GP was immunogenic and efficacious against challenge with a lethal dose of a low-passage MARV Angola. In brief, six groups of cynomolgus macaques (n=4 per group) were vaccinated once by IM injection with doses ranging from 2×10² to 2×10⁷ PFUs of rVSVΔG-MARV-GP. The control cohort received an IM injection with 2×10⁷ PFUs of another rVSVΔG-based vaccine encoding the Lassa virus glycoprotein (rVSVΔG-LASV-GPC) for which Applicants also have developed a preMVS (data not shown) from a promising research vaccine [30]. Following vaccination, samples were collected (FIG. 4A) for measurement of immunogenicity and the macaques were then challenged 28 days post vaccination with a lethal dose of MARV Angola and monitored for clinical signs of MARV disease (FIG. 4B).

As illustrated in the plot in FIG. 4B, all animals vaccinated with the control rVSVΔG-LASV-GPC vaccine developed MARV disease symptoms [20] and were euthanized at days 8 or 9 based on humane endpoint clinical scoring sheet. The progression of MARV disease in the control animals indicated that an anti-VSV vector immune response did not interfere with MARV challenge. However, all animals vaccinated with rVSVΔG-MARV-GP (FIG. 4A) survived (FIG. 4B) and did not develop clinical features of MARV disease.

To assess how vaccination affected the presence of infectious MARV in peripheral blood following challenge, Applicants quantified titers of MARV in serum by plaque assay (FIG. 4C). Determining the titers of infectious virus is important because quantifying RNA copies by RT-qPCR does not measure viable virus progeny circulating in the blood, and importantly, an informative earlier preclinical study has shown that protection from Ebola virus disease progression in macaques was associated with maintaining an infectious titer in blood that was below a threshold titer of about 1×10⁵ tissue culture infectious dose 50 (TCID50) [40]. When Applicants conducted the MARV plaque assay, viremia was detectable in samples collected on day 4 following challenge in all control animals and titers increased to 10⁶ PFUs per ml or more by day 7. At the time of euthanasia per protocol, titers were high at about 108 PFUs/ml (FIG. 4C). In contrast, infectious MARV was undetectable in serum at any time in animals vaccinated with high or low doses of VSVΔG-MARV-GP.

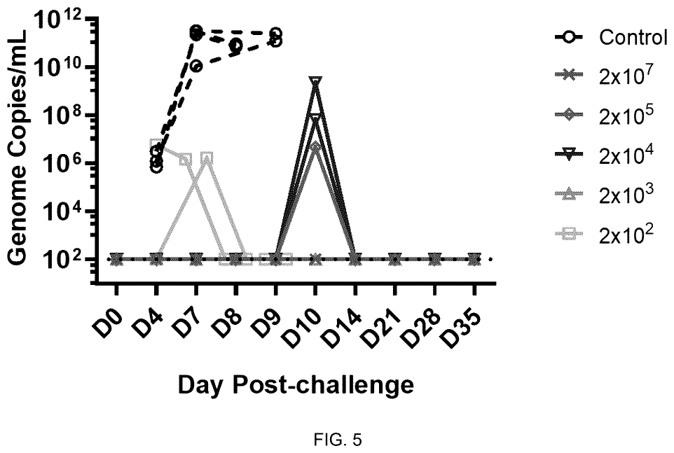

MARV RNA copies also were evaluated RT-qPCR using RNA extracted from whole blood (FIG. 5). As expected based on viral titers in serum, RNA copies in control macaques were high and increased in parallel with titers of infectious virus (FIG. 4C). Consistent with vaccination preventing detectable viremia, MARV RNA was detectable in just 5 of 20 vaccinated animals at any timepoint analyzed after challenge, and if RNA was detected, it was transient (FIG. 5). For example, the 2 animals in the group vaccinated with the lowest dose of 2×10² PFUs of VSVΔG-MARV-GP had transient signal in the RT-qPCR assay. One of these animals was positive on day 4 and 7 but resolved by day 10 while the other was positive only at a single timepoint on day 7. These RNA signals did not increase as would be expected if there was substantial MARV replication occurring in these vaccinated macaques, and it further implied that defective virions might have contributed to the transient RNA signal like those that have been detected before during filovirus infection [41-43]. There also was transient RNA copies present in 2 animals from the group vaccinated with 2×10⁴ PFUs and 1 in the group vaccinated with 2×10⁵ PFUs that was detected at day 10 after challenge (FIG. 5). The presence of a low transient RT-qPCR signal at day 4 or 7 after challenge might be indicative of a MARV infection that was rapidly controlled or aborted resulting in little infectious virus be released into circulation consistent with the viremia data in FIG. 4C. The explanation for a transient RNA signal at day 10 in 3 animals is more speculative particularly since the RNA copies were detected late and did not correlate with the presence of infectious virus (FIG. 4C), but perhaps this is related to clearance of the initial virus inoculum rather than progeny virions produced by active MARV replication. Overall, the analysis RNAemia was consistent with viremia, which both indicated that vaccination prevented significant MARV replication and release of infectious viral progeny into the blood.

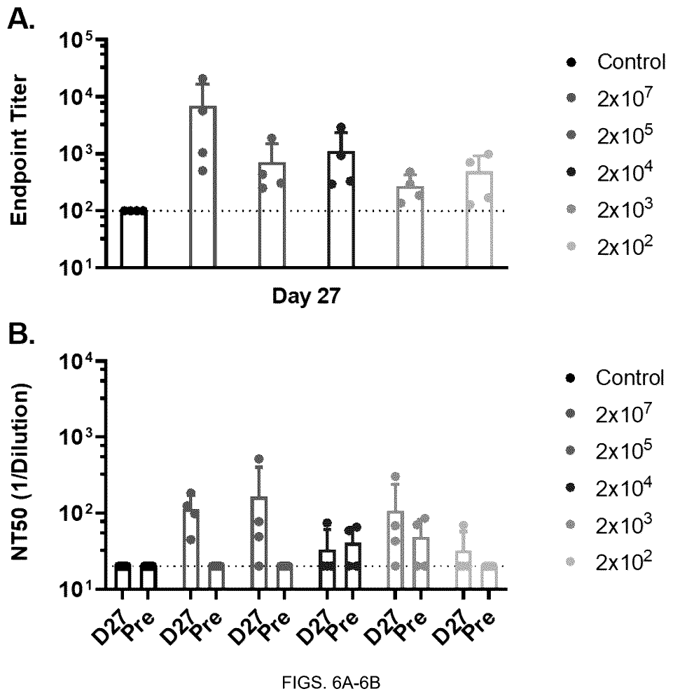

Humoral immune responses against MARV GP induced by VSVΔG-MARV-GP vaccination. Serum was collected (FIG. 4A) from all animals prior to and following vaccination to assess development of anti-GP serum IgG. Binding antibody titers at day 27 just prior to MARV challenge (FIG. 6A) were quantified using ELISA plates coated with a soluble form of GP from the Angola strain. Binding antibodies against the Angola GP were detectable in all animals indicating that a single vaccination, even with the lower doses, resulted in seroconversion. Median titers were highest in the group vaccinated with 2×10⁷ PFUs and generally decreased in proportion to the reduced doses that were tested. The median titers in all groups were statistically significant when compared to the control group vaccinated with VSVΔG-LASV-GPC except for the group vaccinated with just 200 PFUs of VSVΔG-MARV-GP.

Applicants also analyzed serum for virus neutralizing activity (FIG. 6B) using a plaque reduction assay based on neutralization of VSVΔG-MARV-GP (Musoke). The same type of VSV-based assay has been used before to assess neutralizing antibodies against EBOV GP [44-46]. Although the neutralizing titers were low (GMT ˜100 in the high-dose group), this assay did detect neutralizing serum antibodies in 6 of 7 animals vaccinated with the higher vaccine doses (FIG. 3B; 2×10⁷ and 2×10⁵ PFUs). Neutralizing serum antibodies also were detected in some animals vaccinated with lower doses, but low pre-vaccination background neutralization activity also was observed in some of the macaques from these groups.

The replication-competent VSVΔG-MARV-GP vaccine has been shown to be safe and highly efficacious in earlier preclinical studies [16, 22-24, 36-39]. Applicants have developed a new recombinant virus and pre-MVS under conditions that will support production of vaccine material for use in human trials. Applicants also have replicated previous preclinical efficacy results with their vaccine candidate as well as generated important new information. The potential for a particular vaccine technology to be dose-sparing is an important consideration to better enable vaccine access because larger quantities of vaccine material can be produced at a lower cost. Applicants included in the preclinical evaluation of the preMVS an investigation of whether the vaccine was efficacious when doses lower than 2×10⁷ PFUs were used in cynomolgus macaques, a well-characterized filovirus disease model [20, 47, 48]. Applicants found that a single dose of as little as 200 PFUs of rVSVΔG-MARV-GP (Musoke) prevented development of clinical signs of MARV disease following challenge with the Angola MARV strain (FIG. 4B) raising the possibility that lower doses might be effective in people.

Applicants' data showing that low doses of rVSVΔG-MARV-GP are efficacious are consistent with an earlier study showing that very low doses of an rVSVΔG-EBOV-GP (Kikwit variant GP) vaccine could prevent Ebola virus disease in cynomolgus macaques [40]. In this study, Marzi et al showed that as little as 1-10 PFUs of rVSVΔG-EBOV-GP could protect from lethal disease. Although the VSVΔG-MARV-GP and VSVΔG-EBOV-GP preclinical studies clearly illustrate that lower doses of rVSVΔG-based filovirus vaccines are highly protective in macaques, human data from clinical studies with ERVEBO® demonstrated a dose-dependent humoral response that starts to decline at a dose of 10⁵ PFUs [49]. However, because of a lack of human efficacy studies conducted with lower doses, it is not possible to determine if the reduced titers of circulating IgG would be correlated with reduce efficacy against EBOV or EVD disease.

In addition to vaccination preventing development of MARV disease, Applicants found that infectious MARV in the blood was undetectable by plaque assay in vaccinated animals. This indicates that immunity induced by VSVΔG-MARV-GP provided a very effective barrier to development of viremia. This is an important finding as the earlier preclinical investigation of dose and efficacy conducted by Marzi et al. with rVSVΔG-EBOV-GP indicated that immune control of EBOV replication that held viremia below a threshold of about 1×10⁵ TCID50 per ml was key to preventing disease progression [40].

The immune responses that correlate with protection against MARV disease are not well understood. Studies in people vaccinated with rVSVΔG-ZEBOV-GP indicate that total serum IgG titers as well as neutralizing antibody titers against the vaccine virus provide some of the stronger correlates of protection in humans [46]. The potential importance of vaccination inducing functional antibodies might be emphasized by a study showing that antibodies with direct virus-neutralizing activity have been isolated from people vaccinated with VSVΔG-ZEBOV-GP [50, 51], but it is also likely that antibodies capable of mediating Fc-directed innate immune effector functions also play a role in protection from EBOV disease [52]. Thus, it was encouraging that Applicants were able to detect some direct virus neutralization activity specific for the MARV GP in serum from macaques vaccinated with the higher doses of VSVΔG-MARV-GP and that even at the lowest vaccine doses serum IgG titers were detectable. Thus, it will be informative to assess the functional properties of the anti-MARV anti-sera in more detail particularly as innate immune effector functions mediated by the antibody Fc domain are thought to play important roles in protection from MARV disease [53] and it is known that neutralizing monoclonal antibodies specific for the MARV GP are protective [54, 55].

The inability to conduct traditional efficacy studies for pathogens that infect humans sporadically such as highly virulent filoviruses is a challenge for vaccine developers. Thus, to advance a vaccine like VSVΔG-MARV-GP as a product for use in people will require novel regulatory strategies that rely more heavily on animal efficacy models, a detailed understanding of the protective immune response profile induced in animals, and prior experience with a licensed vaccine such as ERVEBO® which uses the same technology [14]. Although there is growing evidence that IgG titers and neutralizing antibodies are correlated with protective immunity induced by ERVEBO® [46], the humoral responses induced by rVSVΔG-MARV-GP are modest, yet vaccination is highly protective in macaques even with low vaccine doses. Thus, to support advancement of future VSVΔG-based filovirus vaccines like rVSVΔG-MARV-GP through regulatory agency approval, it will be important to conduct additional research aimed at a greater understanding of the functional anti-viral glycoprotein adaptive immune responses that contribute to protection and develop an immunologic profile associated with VSVΔG-based vaccine take and efficacy. This could be established through bridging of human clinical data from an effective vaccine like ERVEBO® and preclinical and clinical data from a different VSV filovirus vaccine candidate.

To enable future access to filovirus vaccines, the rVSVΔG-MARV-GP candidate was produced and evaluated in a manufacturing-ready form and is currently being used for production of clinical trial material. Without available clinical trial material for rVSVΔG-EBOV-GP in 2014, it is unlikely that the international community would have an efficacious EBOV vaccine to combat continued EBOV emergence and circulation in Central and Western Africa [56, 57]. Even with this material, international and national public health entities such as the WHO, NIH and CDC, vaccine developers, and public health authorities and experts on the ground in W. Africa needed unprecedented amounts of coordination, collaboration, communication, and clinical capacity to administer and evaluate investigational vaccines during a public health emergency [58]. This 2014-2016 EBOV outbreak in W. Africa allowed for enough data collection to assess the human efficacy of the rVSVΔG-EBOV-GP vaccine. However, the vaccine was used in an expanded access capacity for several years in West and Central Africa before the vaccine was fully licensed in 2019. Important lessons from rVSVΔG-EBOV-GP experience should be applied to prevent future outbreaks of MARV, SUDV, or other filoviruses. Stockpiles urgently need to be generated for investigational vaccines in order to prevent unnecessary future human morbidity and mortality in areas where there is limited human disease in circulation but where the potential impact of outbreaks, should they occur, is large.

The recent MARV cases in Guinea and Ghana underscore the need for sustained preparedness efforts such that Applicants are better equipped to address these continually emerging infectious disease pathogens. The rVSV technology has been evaluated for multiple pathogens [59] and ERVEBO® is already licensed in the U.S., Europe, and multiple African nations [60]. Here Applicants demonstrate an efficacious vaccine against another filovirus, MARV, has dose-sparing qualities observed in well-characterized non-human primate models. This vaccine is effective at doses as low as 200 PFUs in cynomolgus macaques like the protection mediated by rVSVΔG-EBOV-GP at lower doses (FIG. 3 and FIG. 4) [40]. If lower doses of the vaccine are shown to be effective, the costs of goods will be less for vaccine production, a larger number of doses can be produced at a lower cost, and this will enable greater access of the vaccine as needed.

Applicants' results highlight the potential value of the VSV vaccine technology for another important filovirus disease. The similarity between ERVEBO® and this rVSVΔG-MARV-GP vaccine in initial characterization and preclinical data provide a strong foundation for the stockpiling of GMP material for the inclusion in emergency responses for MARV outbreaks. To effectively preposition rVSVΔG-MARV-GP as a public health tool against future MARV outbreaks, stockpiles of clinical trial material need to be generated, similarities between immune responses against EBOV vaccination and MARV need to be explored, and clinical trial designs need to be aligned in advance and prioritized for emergency outbreak situations. The relatedness and similar human disease presentation of EBOV, SUDV, MARV, and other filoviruses suggest that common immune responses can be identified as efficacious across the filovirus family that could enable novel pathways to licensable vaccines. If these objectives can be accomplished for MARV, then greater filovirus preparedness will be achieved. The only way to be better prepared for future outbreaks is by increased and planning and execution of appropriate evaluation and data generation on filovirus vaccine candidates during times without active outbreaks; it is critical that Applicants apply the lessons learned and stay vigilant in order to not be caught off guard once again.

### REFERENCES

1. Sun, J., et al., Ebola virus outbreak returns to the Democratic Republic of Congo: An urgent rising concern. Ann Med Surg (Lond), 2022. 79: p. 103958.
2. Keita, A. K., et al., Resurgence of Ebola virus in 2021 in Guinea suggests a new paradigm for outbreaks. Nature, 2021. 597(7877): p. 539-543.
3. WHO. Ebola virus disease-Democratic Republic of the Congo. 2022 [cited 2022 Jul. 8]; Available from: www.who.int/emergencies/disease-outbreak-news/item/2022-DON377.
4. Munster, V. J., et al., Outbreaks in a Rapidly Changing Central Africa-Lessons from Ebola. N Engl J Med, 2018. 379(13): p. 1198-1201.
5. Pigott, D. M., et al., Mapping the zoonotic niche of Marburg virus disease in Africa. Trans R Soc Trop Med Hyg, 2015. 109(6): p. 366-78.
6. Koundouno, F. R. K., L. E.; Faye, M; Renevey, A; Soropogui, B; Ifono, K; Nelson, E. V.; Kamano, A. A.; Tolno, C; Annibaldis, G; Millimono, S. L.; Camara, J; Kourouma, K; Doré, A; Millimouno, T. E.; Tolno, F. M. B.; Hinzmann, J; Soubrier, H; Hinrichs, M; Thielebein, A; Herzer, G; Pahlmann, M; Ki-Zerbo, G. A.; Formenty, P; Legand, A; Wiley, M. R.; Faye, O; Diagne, M. M.; Sall, A. A.; Lemey, P; Bah, A; Günther, S; Keita, S; Duraffour, S; Magassouba, N., Detection of Marburg Virus Disease in Guinea. NEJM, 2022. 386(26): p. 2528-2530.
7. WHO. Ghana reports first-ever suspected cases of Marburg virus disease. 2022 [cited 2022 Jul. 12]; Available from: www.afro.who.int/countries/ghana/news/ghana-reports-first-ever-suspected-cases-marburg-virus-disease.
8. Carlson, C. J., et al., Climate change increases cross-species viral transmission risk. Nature, 2022.
9. Tell, J. G., et al., Environmental Risk Assessment for rVSVDeltaG-ZEBOV-GP, a Genetically Modified Live Vaccine for Ebola Virus Disease. Vaccines (Basel), 2020. 8(4): p. 10.3390/vaccines8040779.
10. Wolfe, D. N., M. J. Taylor, and A. G. Zarrabian, Lessons learned from Zaire ebolavirus to help address urgent needs for vaccines against Sudan ebolavirus and Marburg virus. Hum Vaccin Immunother, 2020. 16(11): p. 2855-2860.
11. Wolf, J., et al., Development of Pandemic Vaccines: ERVEBO Case Study. Vaccines (Basel), 2021. 9(3).
12. Santoro, F., et al., Human Transcriptomic Response to the VSV-Vectored Ebola Vaccine. Vaccines (Basel), 2021. 9(2).
13. Pinski, A. N. and I. Messaoudi, To B or Not to B: Mechanisms of Protection Conferred by rVSV-EBOV-GP and the Roles of Innate and Adaptive Immunity. Microorganisms, 2020. 8(10).
14. Finch, C. L., et al., Vaccine Licensure in the Absence of Human Efficacy Data. Vaccines (Basel), 2022. 10(3).
15. Dulin, N., et al., Systematic review of Marburg virus vaccine nonhuman primate studies and human clinical trials. Vaccine, 2021. 39(2): p. 202-208.
16. Geisbert, T. W. and H. Feldmann, Recombinant vesicular stomatitis virus-based vaccines against Ebola and Marburg virus infections. J Infect Dis, 2011. 204 Suppl 3: p. S1075-81.
17. Fathi, A., C. Dahlke, and M. M. Addo, Recombinant vesicular stomatitis virus vector vaccines for WHO blueprint priority pathogens. Hum Vaccin Immunother, 2019. 15(10): p. 2269-2285.
18. WHO. A WHO Strategic Agenda for Filovirus Research and Monitoring (AFIRM)-Roadmap Meeting. 2022 [cited 2022 Jul. 8]; Available from: www.who.int/news-room/events/detail/2022/03/30/default-calendar/save-the-date-a-who-strategic-agenda-for-filovirus-research-and-monitoring-(afirm)---roadmap-meeting.
19. Dean, N. E. and I. M. Longini, The ring vaccination trial design for the estimation of vaccine efficacy and effectiveness during infectious disease outbreaks. Clin Trials, 2022: p. 17407745211073594.
20. Glaze, E. R., et al., A Comparison of the Pathogenesis of Marburg Virus Disease in Humans and Nonhuman Primates and Evaluation of the Suitability of These Animal Models for Predicting Clinical Efficacy under the ‘Animal Rule’. Comp Med, 2015. 65(3): p. 241-59.
21. Garbutt, M., et al., Properties of replication-competent vesicular stomatitis virus vectors expressing glycoproteins of filoviruses and arenaviruses. J Virol, 2004. 78(10): p. 5458-65.
22. Daddario-DiCaprio, K. M., et al., Cross-protection against Marburg virus strains by using a live, attenuated recombinant vaccine. J Virol, 2006. 80(19): p. 9659-66.
23. Jones, S. M., et al., Live attenuated recombinant vaccine protects nonhuman primates against Ebola and Marburg viruses. Nat Med, 2005. 11(7): p. 786-90.
24. Mire, C. E., et al., Recombinant vesicular stomatitis virus vaccine vectors expressing filovirus glycoproteins lack neurovirulence in nonhuman primates. PLoS Negl Trop Dis, 2012. 6(3): p. e1567.
25. Espeseth, A. S., et al., Preclinical immunogenicity and efficacy of a candidate COVID-19 vaccine based on a vesicular stomatitis virus-SARS-CoV-2 chimera. EBioMedicine, 2022. 82: p. 104203.
26. Nyombayire, J., et al., First-in-Human Evaluation of the Safety and Immunogenicity of an Intranasally Administered Replication-Competent Sendai Virus-Vectored HIV Type 1 Gag Vaccine: Induction of Potent T-Cell or Antibody Responses in Prime-Boost Regimens. J Infect Dis, 2017. 215(1): p. 95-104.
27. Witko, S. E., et al., Refined methods for propagating vesicular stomatitis virus vectors that are defective for G protein expression. J Virol Methods, 2010. 164(1-2): p. 43-50.
28. Witko, S. E., et al., An efficient helper-virus-free method for rescue of recombinant paramyxoviruses and rhadoviruses from a cell line suitable for vaccine development. J Virol Methods, 2006. 135(1): p. 91-101.
29. Rabinovich, S., et al., A novel, live-attenuated vesicular stomatitis virus vector displaying conformationally intact, functional HIV-1 envelope trimers that elicits potent cellular and humoral responses in mice. PLoS One, 2014. 9(9): p. e106597.
30. Geisbert, T. W., et al., Development of a new vaccine for the prevention of Lassa fever. PLoS Med, 2005. 2(6): p. e183.
31. Woolsey, C., et al., Postexposure Efficacy of Recombinant Vesicular Stomatitis Virus Vectors Against High and Low Doses of Marburg Virus Variant Angola in Nonhuman Primates. J Infect Dis, 2018. 218(suppl_5): p. 5582-5587.
32. Woolsey, C., et al., Immune correlates of postexposure vaccine protection against Marburg virus. Sci Rep, 2020. 10(1): p. 3071.
33. Boritz, E., et al., Replication-competent rhabdoviruses with human immunodeficiency virus type 1 coats and green fluorescent protein: entry by a pH-independent pathway. J Virol, 1999. 73(8): p. 6937-45.
34. Schnell, M. J., et al., The minimal conserved transcription stop-start signal promotes stable expression of a foreign gene in vesicular stomatitis virus. J Virol, 1996. 70(4): p. 2318-23.
35. Lawson, N. D., et al., Recombinant vesicular stomatitis viruses from DNA. Proc Natl Acad Sci USA, 1995. 92(10): p. 4477-81.
36. Geisbert, T. W., et al., Postexposure treatment of Marburg virus infection. Emerg Infect Dis, 2010. 16(7): p. 1119-22.
37. Daddario-DiCaprio, K. M., et al., Postexposure protection against Marburg haemorrhagic fever with recombinant vesicular stomatitis virus vectors in non-human primates: an efficacy assessment. Lancet, 2006. 367(9520): p. 1399-404.
38. Mire, C. E., et al., Durability of a vesicular stomatitis virus-based marburg virus vaccine in nonhuman primates. PLoS One, 2014. 9(4): p. e94355.
39. Geisbert, T. W., et al., Vesicular stomatitis virus-based vaccines protect nonhuman primates against aerosol challenge with Ebola and Marburg viruses. Vaccine, 2008. 26(52): p. 6894-900.
40. Marzi, A., et al., Single low-dose VSV-EBOV vaccination protects cynomolgus macaques from lethal Ebola challenge. EBioMedicine, 2019. 49: p. 223-231.
41. Calain, P., L. Roux, and D. Kolakofsky, Defective interfering genomes and Ebola virus persistence. Lancet, 2016. 388(10045): p. 659-60.
42. Johnson, R. I., et al., Identification and Characterization of Defective Viral Genomes in Ebola Virus-Infected Rhesus Macaques. J Virol, 2021. 95(17): p. e0071421.
43. Smither, S. J., et al., An Investigation of the Effect of Transfected Defective, Ebola Virus Genomes on Ebola Replication. Front Cell Infect Microbiol, 2020. 10: p. 159.
44. Konduru, K., et al., High degree of correlation between Ebola virus BSL-4 neutralization assays and pseudotyped VSV BSL-2 fluorescence reduction neutralization test. J Virol Methods, 2018. 254: p. 1-7.
45. Wilkinson, D. E., et al., Comparison of platform technologies for assaying antibody to Ebola virus. Vaccine, 2017. 35(9): p. 1347-1352.
46. Grais, R. F., et al., Estimation of the correlates of protection of the rVSVΔG-ZEBOV-GP Zaire ebolavirus vaccine: a post-hoc analysis of data from phase 2/3 clinical trials. The Lancet Microbe, 2021. 2(2): p. e70-e78.
47. Niemuth, N. A., et al., Natural history of disease in cynomolgus monkeys exposed to Ebola virus Kikwit strain demonstrates the reliability of this non-human primate model for Ebola virus disease. PLoS One, 2021. 16(7): p. e0252874.
48. Woolsey, C., et al., Natural history of Sudan ebolavirus infection in rhesus and cynomolgus macaques. Emerg Microbes Infect, 2022. 11(1): p. 1635-1646.
49. Huttner, A., et al., The effect of dose on the safety and immunogenicity of the VSV Ebola candidate vaccine: a randomised double-blind, placebo-controlled phase 1/2 trial. Lancet Infect Dis, 2015. 15(10): p. 1156-66.
50. Saphire, E. O., A glimpse into immune responses evolving against Ebola virus. Nat Med, 2019. 25(10): p. 1470-1471.
51. Ehrhardt, S. A., et al., Polyclonal and convergent antibody response to Ebola virus vaccine rVSV-ZEBOV. Nat Med, 2019. 25(10): p. 1589-1600.
52. Saphire, E. O., et al., Antibody-mediated protection against Ebola virus. Nat Immunol, 2018. 19(11): p. 1169-1178.
53. Ilinykh, P. A., et al., Non-neutralizing Antibodies from a Marburg Infection Survivor Mediate Protection by Fc-Effector Functions and by Enhancing Efficacy of Other Antibodies. Cell Host Microbe, 2020. 27(6): p. 976-991 e11.
54. Mire, C. E., et al., Therapeutic treatment of Marburg and Ravn virus infection in nonhuman primates with a human monoclonal antibody. Sci Transl Med, 2017. 9(384).
55. Brannan, J. M., et al., Post-exposure immunotherapy for two ebolaviruses and Marburg virus in nonhuman primates. Nat Commun, 2019. 10(1): p. 105.
56. Coller, B. G., et al., Clinical development of a recombinant Ebola vaccine in the midst of an unprecedented epidemic. Vaccine, 2017. 35(35 Pt A): p. 4465-4469.
57. Regules, J. A., et al., A Recombinant Vesicular Stomatitis Virus Ebola Vaccine. N Engl J Med, 2017. 376(4): p. 330-341.
58. Gupta, S. B., B. A. Coller, and M. Feinberg, Unprecedented pace and partnerships: the story of and lessons learned from one Ebola vaccine program. Expert Rev Vaccines, 2018. 17(10): p. 913-923.
59. Liu, G., et al., Vesicular Stomatitis Virus: From Agricultural Pathogen to Vaccine Vector. Pathogens, 2021. 10(9).
60. Malenfant, J. H., et al., Use of Ebola Vaccine: Expansion of Recommendations of the Advisory Committee on Immunization Practices To Include Two Additional Populations-United States, 2021. MMWR Morb Mortal Wkly Rep, 2022. 71(8): p. 290-292.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A vaccine comprising a nucleic acid encoding a Musoke isolate Marburg virus (MARV) glycoprotein (GP) encoded in a vesicular stomatitis vector (VSV) excluding a glycoprotein G gene (VSVΔG), and a pharmaceutically acceptable carrier.

2. The vaccine of claim 1, wherein the nucleic acid comprises SEQ ID NO: 1.

3. The vaccine of claim 1, wherein the nucleic acid comprises SEQ ID NO: 2.

4. The vaccine of claim 1, wherein the nucleic acid comprises a sequence having at least 95% sequence identity to SEQ ID NO: 1, or at least 95% sequence identity to SEQ ID NO: 2.

5. The vaccine of claim 1, wherein the pharmaceutically acceptable carrier comprises a stabilizing excipient comprising sucrose, trehalose, or sodium chloride.

6. The vaccine of claim 1, wherein the nucleic acid encoding the MARV GP is codon-optimized for expression in a human.

7. The vaccine of claim 1, wherein the vaccine elicits an immune response in a primate against MARV Angola, MARV Musoke, MARV Angola, or Ravn virus.

8. The vaccine of claim 1, comprising about 10² to 10⁷ plaque-forming units (PFU) of the VSV per dose.

9. The vaccine of claim 8, wherein the PFU is 10⁴ PFU or 10⁵ PFU or 10⁶ PFU of the VSV per dose.

10. The vaccine of claim 1, wherein the pharmaceutically acceptable carrier provides that the vaccine is formulated as a sterile injectable solution or dispersion suitable for intramuscular, intranasal, or intradermal administration.

11. The vaccine of claim 1, wherein the pharmaceutically acceptable carrier provides that the vaccine is formulated as an oral bait drop.

12. The vaccine of claim 11, wherein the oral bait drop is suitable for administration to a bat or a primate.

13. The vaccine of claim 1, wherein administration of the vaccine to a mammal elicits antibodies that mediate Fc-dependent immune effector functions.

14. The vaccine of claim 1, wherein a single dose of the vaccine elicits a protective immune response in a primate against MARV.

15. The vaccine of claim 1, wherein the nucleic acid encoding the MARV GP replaces the genomic locus of the VSV glycoprotein G gene.

16. A pharmaceutical composition comprising a nucleic acid encoding a Musoke isolate Marburg virus (MARV) glycoprotein (GP) encoded in a vesicular stomatitis vector (VSV) excluding a glycoprotein G gene (VSVΔG). a pharmaceutically acceptable carrier.

17. The pharmaceutical composition of claim 16, wherein the pharmaceutical composition is formulated as a unit dosage form comprising about 10² to 10⁷ plaque-forming units (PFU) of the vaccine, or for intramuscular, intranasal, or intradermal administration.

18. The pharmaceutical composition of claim 16, wherein the nucleic acid comprises SEQ ID NO:1, or a sequence having at least 95% sequence identity to SEQ ID NO: 1, or SEQ ID NO:2, or a sequence having at least 95% sequence identity to SEQ ID NO:2.

19. The pharmaceutical composition of claim 16, wherein the pharmaceutically acceptable carrier comprises a stabilizing excipient comprising sucrose, trehalose, or sodium chloride.

20. A combination vaccine comprising the vaccine of claim 1 and a second vaccine comprising an Ebola virus vaccine or a Sudan virus vaccine.
